SEQUENCE LISTING (2 sequences)

SEQ ID NO: 1 - DNA, 2574 bp, Homo sapiens
  gene: described in examples and illustrated in Figure 1
gcgtcgacagcaggacaagctcagcctgcagctgccgtgggctttgtgtggactggacgcagagcttgggagacgggggagggctattactccaattcactgtcaatggaattacagctatagcggcagtgtatataggattgctttttctcgtcttcctgggttctgaagtaacggaagctaccttgtataaagacctcaacactgctgaccatgatcagcgcagcctggagcatcttcctcatcgggactaaaattgggctgttccttcaagtagcacctctatcagttatggctaaatcctgtccatctgtgtgtcgctgcgatgcgggtttcatttactgtaatgatcgctttctgacatccattccaacaggaataccagaggatgctacaactctctaccttcagaacaaccaaataaataatgctgggattccttcagatttgaaaaacttgctgaaagtagaaagaatatacctataccacaacagtttagatgaatttcctaccaacctcccaaagtatgtaaaagagttacatttgcaagaaaataacataaggactatcacttatgattcactttcaaaaattccctatctggaagaattacatttagatgacaactctgtctctgcagttagcatagaagagggagcattccgagacagcaactatctccgactgcttttcctgtcccgtaatcaccttagcacaattccctggggtttgcccaggactatagaagaactacgcttggatgataatcgcatatccactatttcatcaccatctcttcaaggtctcactagtctaaaacgcctggttctagatggaaacctgttgaacaatcatggtttaggtgacaaagttttcttcaacctagttaatttgacagagctgtccctggtgcggaattccctgactgctgcaccagtaaaccttccaggcacaaacctgaggaagctttatcttcaagataaccacatcaatcgggtgcccccaaatgctttttcttatctaaggcagctctatcgactggatatgtccaataataacctaagtaatttacctcagggtatctttgatgatttggacaatataacacaactgattcttcgcaacaatccctggtattgcgggtgcaagatgaaatgggtacgtgactggttacaatcactacctgtgaaggtcaacgtgcgtgggctcatgtgccaagccccagaaaaggttcgtgggatggctattaaggatctcaatgcagaactgtttgattgtaaggacagtgggattgtaagcaccattcagataaccactgcaatacccaacacagtgtatcctgcccaaggacagtggccagctccagtgaccaaacagccagatattaagaaccccaagccactaaggatcaccaaaccacagggagtccctcaagaaaaacaattacaattactgtgaagtctgtcacttctgataccattcatatctcttggaaacttgctctacctatgactgctttgagactcagctggcttaaactgggccatagcccggcatttggatctataacagaaacaattgtaacaggggaacgcagtgagtacttggtcacagccctggagcctgattcaccctataaagtatgcatggttcccatggaaaccagcaacctctacctatttgatgaaactcctgtttgtattgagactgaaactgcaccccttcgaatgtacaaccctacaaccaccctcaatcgagagcaagagaaagaaccttacaaaaaccccaatttacctttggctgccatcattggtggggctgtggccctggttaccattgcccttcttgctttagtgtgttggtatgttcataggaatggatcgctcttctcaaggaactgtgcatatagcaaagggaggagaagaaaggatgactatgcagaagctggcactaagaaggacaactctatcctggaaatcagggaaacttcttttcagatgttaccaataagcaatgaacccatctcgaaggaggagtttgtaatacacaccatatttcctcctaatggaatgaatctgtacaaaaacaatcacagtgaaagcagtagtaaccgaagctacagagacagtggtattccagactcagatcactcacactcatgatgctgaaggactcacagcagacttgtgttttgggttttttaaacctaagggaggtgatggtaggaaccctgttctactgcaaaacactggaaaaagagactgaaaaaaagcaatgtactgtacatttgccatataatttatatttaagaactttttattaaaagtttcaaatttcaggttactgctgccgattgatgtagtggagatgcctgaacacaattctatatttagtatttttttagtaattgtactgtattttccttgcaaatattggagttataaaccatttactttgtgttctactgagtaagatgacttgttgactgtgaaagtgaattttcttgctgtgtcgaacaatcaggactgcattcatatgagatccttgtagtataagcacaggccatttttcagt

SEQ ID NO: 2 - protein, 649 aa, Homo sapiens
  peptide
MISAAWSIFLIGTKIGLFLQVAPLSVMAKSCPSVCRCDAGFIYCNDRFLTSIPTGIPEDATTLYLQNNQINNAGIPSDLKNLLKVERIYLYHNSLDEFPTNLPKYVKELHLQENNIRTITYDSLSKIPYLEELHLDDNSVSAVSIEEGAFRDSNYLRLLFLSRNHLSTIPWGLPRTIEELRLDDNRISTISSPSLQGLTSLKRLVLDGNLLNNHGLGDKVFFNLVNLTELSLVRNSLTAAPVNLPGTNLRKLYLQDNHINRVPPNAFSYLRQLYRLDMSNNNLSNLPQGIFDDLDNITQLILRNNPWYCGCKMKWVRDWLQSLPVKVNVRGLMCQAPEKVRGMAIKDLNAELFDCKDSGIVSTIQITTAIPNTVYPAQGQWPAPVTKQPDIKNPKLTKDHQTTGSPSRKTITITVKSVTSDTIHISWKLALPMTALRLSWLKLGHSPAFGSITETIVTGERSEYLVTALEPDSPYKVCMVPMETSNLYLFDETPVCIETETAPLRMYNPTTTLNREQEKEPYKNPNLPLAAIIGGAVALVTIALLALVCWYVHRNGSLFSRNCAYSKGRRRKDDYAEAGTKKDNSILEIRETSFQMLPISNEPISKEEFVIHTIFPPNGMNLYKNNHSESSSNRSYRDSGIPDSDHSHS

(19) United States
(12) Patent Application Publication
(10) Pub. No.: US 2003/0215884 A1
(43) Pub. Date: Nov. 20, 2003

(54) Method of regenerating neurons

(76) Inventors: Stephen P. Hunt, London (GB); Michelle Robinson, London (GB); Frederick Livesey, Cambridge (GB)

Correspondence Address: MARY M. KRINSKY, Ph. D., J.D., PATENT ATTORNEY, 79 TRUMBULL STREET, NEW HAVEN, CT, 06511, US

(21) Appl. No.: 10/145,541

(22) Filed: May 14, 2002

(51) Int. Cl. G01N 33/53; G01N 33/567; A61K 38/17

(52) U.S. Cl. 435/7.2; 514/12

(57) ABSTRACT

The invention relates to a method of regenerating neurons comprising administering to a subject in need thereof FLRT-3 to cause neuronal regeneration.

### FIELD OF INVENTION

The present invention relates to methods for regenerating neurons, assay methods and products derived therefrom.

In particular, the present invention relates to a method of regenerating neurons comprising administering to a subject in need thereof FLRT-3 to cause neuronal regeneration.

### BACKGROUND TO THE INVENTION

The failure of neurons to regenerate axons in the damaged brain and spinal cord is a major cause of human suffering in many common neurological disorders such as brain and spinal cord trauma, stroke, cerebral palsy and multiple sclerosis.

Axonal regeneration in injured peripheral nerves is vigorous and often leads to functional recovery. In contrast, axonal regeneration in the central nervous system (CNS) of adult mammals is normally minimal and the consequences of injury to the brain and spinal cord are often severe and prolonged. Although various ways have been discovered to bring about the limited regeneration of some classes of axons in the CNS, the reasons why regeneration normally fails are still poorly understood. Both the presence of inhibitory molecules in the CNS and the poor intrinsic response to axotomy of many CNS neurons has been implicated in the failure of regeneration, but the precise role of individual molecules is obscure.

A large number of molecules and epitopes capable of inhibiting axonal regeneration have been identified in the CNS. These include axonal guidance molecules, such as semaphorins, which have inhibitory functions during development and interact with specific receptors on particular classes of neurons (Pasterkamp et al., 2001; Liu and Strittmatter, 2001); extracellular matrix molecules with more general inhibitory effects, such as tenascin-C and some chondroitin sulphate proteoglycans (McKeon et al., 1991; Pasterkamp et al., 2001; Moon et al., 2001); and molecules such as NG2 and MAG, found on the surfaces of particular classes of glia (Fidler et al., 1999; Qia et al., 2000). NogoA, a major growth-inhibitory molecule in CNS myelin (Fouad et al., 2001), appears to be largely an oligodendrocyte protein and interacts with a specific receptor on neurons (Fournier et al., 2001; Skaper et al., 2001). While it is not entirely clear how inhibitory the environment in the intact CNS is to axonal growth, there is both direct and circumstantial evidence that some inhibitory molecules are upregulated in and around sites of injury to the brain and spinal cord. The growth inhibitory properties of CNS lesion sites seem to be related to the glial response and the inflammatory response to injury (Moon et al., 2001; Ramer et al., 2001).

There is now abundant evidence that the vigour of axonal regeneration varies dramatically, depending on the nature and site of axonal injury and on the type of neuron that is axotomised (de Felipe et al., 1993, Anderson et al., 1998; Bradbury et al., 2000). For example, the central processes of dorsal root ganglion cells regenerate much less vigorously than the peripheral process, and this can be correlated with the very limited changes in gene expression in sensory neurons after a central, as opposed to a peripheral axotomy (Jenkins et al., 1993a). Similarly, intrinsic CNS neurons show marked differences in their capacity to regenerate axons into peripheral nerve grafts implanted into the brain or spinal cord (Hull and Bähr, 1994; Chaikunksunt et al., 2000a,b). Once again, the capacity to regenerate vigorously depends on the pattern of gene expression in the injured CNS neurons; some neurons fail to express growth-related genes and such cells fail to regenerate axons (Anderson et al., 1998). Even in the regenerating peripheral nervous system, there is frequent and substantial loss in the force and innate precision of neurological function and muscle atrophy even after recovery, compared to the situation before injury, which could be improved by a faster axonal regeneration (Ishii et al., 1994; Gold et al., 1999).

The present invention seeks to provide methods for the regeneration of neurons.

### SUMMARY OF THE INVENTION

The present invention is based upon the surprising finding that FLRT-3 is expressed by damaged sensory neurons and is pro-regenerative in adult peripheral neurons but fails to reappear in central neurons following damage, although it is expressed widely in the CNS during development. Without being limited to any particular theory it is proposed herein that the reintroduction of FLRT-3 may be of use in repairing the damaged CNS.

Thus, in a first aspect, the present invention relates to a method of regenerating neurons comprising administering to a subject in need thereof FLRT-3 to cause neuronal regeneration.

Preferably, FLRT-3 is administered in combination with an agent that modulates FLRT-3.

In a second aspect, the present invention relates to a method of regenerating neurons comprising administering to a subject in need thereof an agent that modulates FLRT-3 to cause neuronal regeneration.

In a third aspect, the present invention relates to an assay method for identifying an agent that modulates the activity of FLRT-3 comprising determining the activity of FLRT-3 in the presence or absence of the agent.

Preferably, the assay method comprises the step of determining if the agent modulates the ability of FLRT-3 to regenerate neurons.

Preferably, the neurons are in the central nervous system.

Preferably, the assay method comprises the additional step of formulating said identified agent as a pharmaceutical compound with said FLRT-3.

In a fourth aspect, the present invention relates to a method comprising the steps of: (a) performing the assay method of the present invention; (b) identifying one or more agents capable of modulating FLRT-3; and (c) preparing a quantity of those one or more identified agents.

In a fifth aspect, the present invention relates to a method comprising the steps of: (a) performing the method according to the present invention; (b) identifying one or more agents capable of modulating FLRT-3; and (c) preparing a pharmaceutical composition comprising those one or more identified agents.

In a sixth aspect, the present invention relates to an agent identified by the assay method of the present invention.

In a seventh aspect, the present invention relates to a pharmaceutical composition prepared by the method of the present invention.

In an eighth aspect, the present invention relates to a method of preventing and/or treating a disease comprising administering an agent according to the present invention and/or a pharmaceutical composition according to the present invention wherein said agent or pharmaceutical composition is capable of modulating the activity of FLRT-3 to cause a beneficial preventative and/or therapeutic effect.

Preferably, the pharmaceutical composition is administered to the central nervous system.

Preferably, the disease is selected from the group consisting of brain trauma, spinal cord trauma, stroke, cerebral palsy, multiple sclerosis, neuronal migration and muscle cell migration and degenerative diseases such as motor neuron disease, Parkinsons disease and Alzheimers disease.

In an eighth aspect, the present invention relates to a non-human animal having a silenced or eliminated or removed FLRT-3 and/or a silenced or eliminated or removed nucleotide sequence coding FLRT-3.

### DESCRIPTION OF THE DRAWINGS

FIG. 1 represents (a) the DNA sequence and (b) the amino acid sequence of human FLRT-3.

FIG. 2 represents a diagrammatic representation of the Northern blot analysis of adult dorsal root ganglia (DRG).

FIG. 3 represents a series of photographs illustrating the in-situ hybridisation analysis of adult rat DRG.

FIG. 4 represents a series of photographs illustrating the in-situ hybridisation analysis of adult rat motor neurons.

FIG. 5 represents a series of photographs illustrating (A) wholemount in-situ hybridisation analysis of E12 rat (B, branchial arch; D, diencephalon; E, eye; FL, forelimb; LD, lip of the dermamyotome; S, somites); (B) in-situ hybridisation analysis in the lateral lip of the dermamyotome (SC, spinal cord) and (C) in-situ hybridisation analysis in the telencephalic vesicle.

FIG. 6 represents a series of photographs illustrating the in-situ hybridisation analysis in (A) adult rat brain (DG, dentate gyrus); and (B) E-17 rat brain (CP, cortical plate; IZ, intermediate zone; VZ, ventricular zone).

FIG. 7 represents a diagrammatic illustration of the structure of FLRT-3 by protein prediction analysis.

FIG. 8 represents a series of photographs illustrating the membrane localisation of FLRT-3.

FIG. 9 represents a series of photographs illustrating double-label immunostaining showing FLRT-3 expression (i) and neurofilament N52 expression (ii) in cultured adult rat DRG neuronal cells; the superimposed image is shown in (iii).

FIG. 10 represents a series of histograms illustrating the antisense knockdown of FLRT-3 protein in cultures of dissociated adult rat DRG resulting in a significant reduction in (i) neurite number and (ii) neurite length.

### DETAILED DESCRIPTION OF THE INVENTION

FLRT-3

FLRT-3 is a member of a novel family of transmembrane leucine-rich repeat proteins originally identified by Lacy et al. (1999) during a search for potential gene candidates for laminin 2-positive congenital muscular dystrophies. The complete coding region of FLRT-3 was isolated following a screen of the human skeletal muscle library. Lacy et al. (1999) have characterised FLRT-3 as comprising 10 leucine-rich repeats (LRRs). The LRR motif was first identified by Takahashi et al. (1985) as a protein motif present in the leucine-rich α₂-glycoprotein. FLRT-3 was found to contain amino and carboxyl LRR cysteine flanks that conform to the published consensus sequences (Buchanan and Gay, 1996; Kobe and Deisenhofer, 1994). Hydropathy plots of the FLRT-3 sequence were used to identify a conserved region of 28 hydrophobic amino acids beginning approximately 100 amino acids from the C-terminal end, representative of a potential membrane-spanning region. Overall, the hydropathy plots suggest that FLRT-3 spans the lipid bilayer once. A putative signal sequence was also found to be present at the amino terminus of FLRT-3.

The present inventors have used differential display of mRNA and cDNA library screening to isolate a gene encoding FLRT-3 that is up-regulated in adult rat DRG 3 days after sciatic nerve crush.

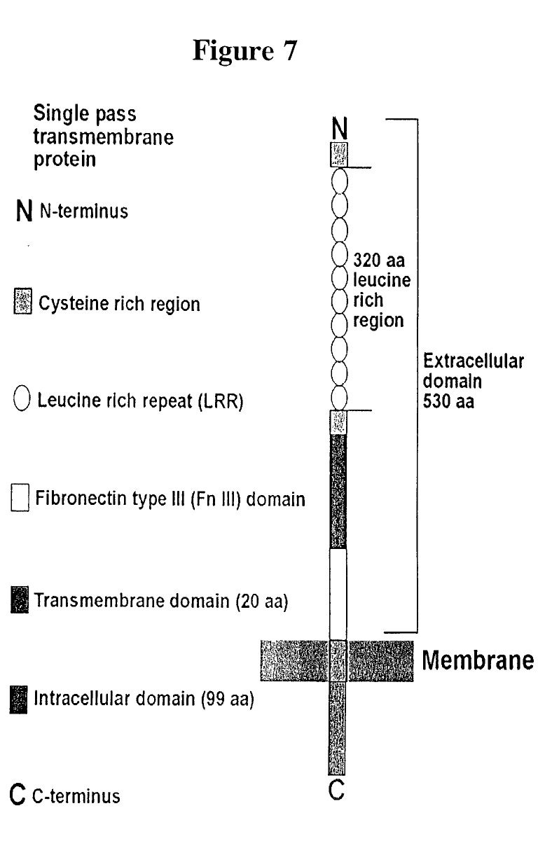

Structure prediction analysis (FIG. 7) suggests that FLRT-3 is a single pass transmembrane protein comprising a 530 amino acid N-terminal extracellular domain, a 20 amino acid transmembrane domain, and a 99 amino acid C-terminal intracellular domain. Much of the extracellular face consists of a 320 amino acid leucine rich region, containing 10 leucine rich repeats, which is flanked by cysteine rich regions. A fibronectin type III domain follows this. The intracellular region contains no recognisable motifs. Without being bound by any particular theory, this suggests that FLRT-3 may be a cell surface receptor or cell adhesion molecule. It is similar in some respects to the NogoA receptor which is a major growth-inhibitory molecule in CNS myelin (Fouad et al., 2001), but there is little homology at the protein or nucleotide level.

Without wishing to be bound by any particular theory, it is proposed herein that FLRT-3 is a receptor and that binding of a ligand modulates—such as increases—the regenerative potential of neurons.

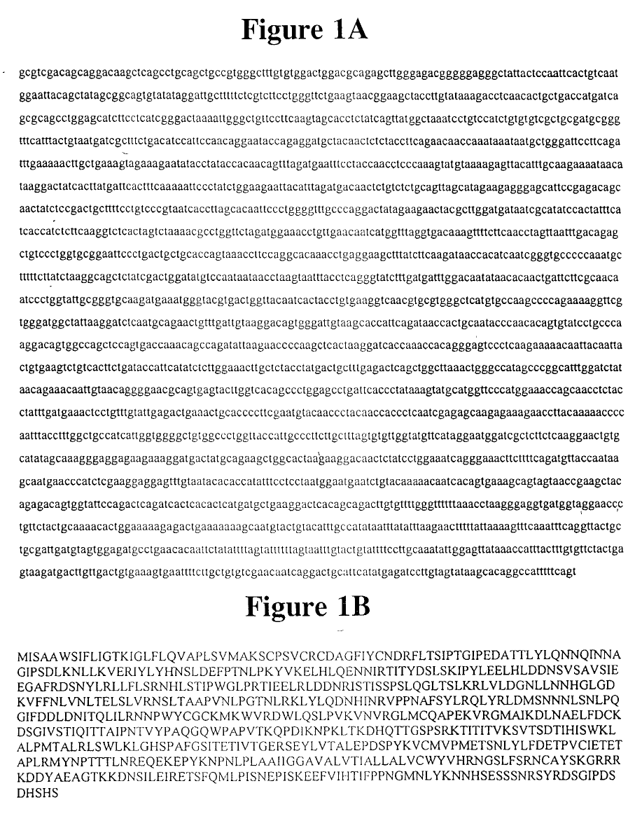

In a highly preferred aspect, FLRT-3 comprises the sequence disclosed in Genbank Accession number AF169677 and detailed in FIG. 1.

As used herein, the term “FLRT-3” also includes fragments, mutants, variants or homologues of FLRT-3 that retain the properties thereof

Regenerating Neurons

The term “regenerating neurons” refers to the regeneration of neurons—such as damaged neurons—to restore neural functions. Neural functions may be partially or completely restored such that at least 10%, 20%, 30%, 40%, 50%, 60%, 70%, 80%, 90% or even substantially 100% of neural functions are restored. Typically, restoration of neural functions may result in an increase in the length of the longest neurite per cell relative to control cultures, increases in the number of neurites per cell, increases in axonal outgrowth and regeneration; hypinnervation and the formation of inappropriate connections.

Various methods known to a person skilled in the art may be used to determine whether neurons have been regenerated—such as anatomical methods (e.g. identifying growth cones and/or myelinated fibres) and behavioural methods (e.g. functional tests, pain sensation tests and/or electromyography).

The neurons to be regenerated may be damaged due to a disease ie. any anatomical abnormality or impairment of the normal functioning of an organism or any parts thereof including those arising directly from physical injury. The disease may be caused by environmental factors, infective agents, inherent defects in the organism or any combination thereof.

In a preferred embodiment, the disease is brain trauma, spinal cord trauma, stroke, cerebral palsy, multiple sclerosis, neuronal migration, muscle cell migration or degenerative diseases such as motor neuron disease, Parkinsons disease and Alzheimers disease.

Preferably, the neurons that are regenerated are neurons of the CNS.

The present inventors have found that FLRT-3 is expressed by all damaged sensory neurons and is pro-regenerative in adult peripheral neurons but fails to reappear in central neurons following damage even though during development the molecule is widely expressed in the CNS. Over-expression of FLRT-3 in cultured dissociated adult rat DRG produces an increase in the length of the longest neurite per cell relative to control cultures and the number of neurites per cell also increases.

A large number of molecules and epitopes capable of inhibiting axonal regeneration have been identified in the CNS. These include axonal guidance molecules, such as semaphorins, which have inhibitory functions during development and interact with specific receptors on particular classes of neurons (Pasterkamp et al., 2001; Liu and Strittmatter, 2001), extracellular matrix molecules with more general inhibitory effects, such as tenascin-C and some chondroitin sulphate proteoglycans (McKeon et al., 1991; Pasterkamp et al., 2001; Moon et al., 2001), and molecules such as NG2 and MAG, found on the surfaces of particular classes of glia (Fidler et al., 1999; Qia et al., 2000). NogoA, a major growth-inhibitory molecule in CNS myelin (Fouad et al., 2001), appears to be largely an oligodendrocyte protein and interacts with a specific receptor on neurons (Fournier et al., 2001; Skaper et al., 2001). While it is not entirely clear how inhibitory the environment in the intact CNS is to axonal growth, there is both direct and circumstantial evidence that some inhibitory molecules are upregulated in and around sites of injury to the brain and spinal cord. The growth inhibitory properties of CNS lesion sites seem to be related to the glial response and the inflammatory response to injury (Moon et al., 2001; Ramer et al., 2001).

Without being limited to any particular theory it is proposed herein that the reintroduction of FLRT-3 may be of use in repairing neurons—such as damaged CNS neurons—by driving axonal outgrowth and/or overcoming inhibitory influences within the external environment.

Administration

Aspects of the present invention relate to the administration of FLRT-3 either alone or in combination with an agent.

Aspects of the present invention also relate to the administration of agents that modulate FLRT-3.

As used herein the term “administration” includes delivery by viral or non-viral techniques.

Non-viral delivery mechanisms include, but are not limited to transfection, lipid mediated transfection, liposomes, immunoliposomes, lipofectin, cationic facial amphiphiles (CFAs) and combinations thereof.

Delivery may be achieved using mechanical pumps to allow delivery directly into the brain parenchyma, for example. Polymer matrices loaded with macromolecules such as proteins allow the sustained delivery in brain parenchyma of animal models (Hoffman et al. (1990) Exp. Neurol. 110, 39-44).

Physical injection of nucleic acid into cells represents the simplest gene delivery system (Vile & Hart (1994) Ann. Oncol, suppl. 5, 59). Accordingly, vectors comprising nucleotide sequences may be administered directly as “a naked nucleic acid construct” and may further comprise flanking sequences homologous to the host cell genome. After injection, nucleic acid is taken into cells and translocated to the nucleus, where it may be expressed transiently from an episomal location or stably if integration into the host genome has occurred. The gene encoding FLRT-3 may be placed under the control of promoters. Physical interventions may increase transfection efficiency, for example, focused ultrasound. The efficiency of transfection of cells in vivo may be increased by injecting DNA coated gold particles with a gene gun (Fyan et al. (1993) Proc. Natl. Acad. Sci. USA 90,11478).

Liposomes are vesicles composed of phospholipid bilayer membranes that can enclose various substances, including nucleic acid. Mixtures of lipids and nucleic acid form complexes (lipoplexes) that can transfect cells in vitro and in vivo. Lipid mediated gene delivery has the ability to transfect various different cells without the need for interaction with specific receptors, minimal immunogenicity of the lipid components to facilitate multiple administration, high capacity vectors with the ability to deliver large DNA sequences and ease of production. The insertion of polyethylene glycol derivatives into the lipid membrane or pegylation may increase the circulation half-life of liposomes after administration. The pharmacokinetics, biodistribution and fusogenicity of liposomes may be varied by altering the composition of the lipid membrane. In particular, the incorporation of certain cationic lipids, for example, DMRIE, DOSPA and DOTAP with neutral or helper co-lipids—such as cholesterol or DOPE—in liposomes may increase their ability to fuse with cell membranes and deliver their contents into cells.

A number of nonlipid polycationic polymers form complexes with nucleic acid which promotes delivery into cells (Li and Huang (2000) Gene Ther. 7, 31). Preferably, the nonlipid polycationic polymers include but are not limited to poly-L-lysine, polyethylenimine, polyglucosamines and peptoids. Polyethylenimine may protect complexed nucleic acids from degradation within endosomes and it also provides a means of promoting nucleic acid release from the endosomal compartment and its subsequent translocation to the nucleus (Boussif et al. (1995) Proc. Natl. Acad. Sci. USA 92, 7297). Pegylated polyethylenimine polymers may decrease the interaction with serum proteins, extended circulation half-life and may deliver genes to cells without significant toxicity.

The transplantation of cells, for example, autologous, allogeneic and xenogeneic cells, that are genetically engineered to release biotherapeutic molecules—such as FLRT-3—may also be used. The transplanted cells may be surrounded with a permselective membrane that fully contains and protects them from attack by the host immune system. This method of encapsulation allows the neural transplantation of primary cells or cell lines from both allogeneic and xenogeneic sources. Various types of encapsulation techniques are known in the art. The method of microencapsulation allows the entrapment of small cell clusters within a thin, spherical, semipermeable membrane typically made of polyelectrolytes.

Viral delivery mechanisms are attractive vehicles for gene delivery since they have evolved specific and efficient means of entering human cells and expressing their genes. Preferably, the viral genome is modified to remove sequences required for viral replication and pathogenicity. More preferably, the viral coding sequences are replaced with exogenous genes—such as FLRT-3.

Viral delivery mechanisms include but are not limited to retrovirus, adenovirus, adeno-associated virus, herpes simplex virus, pox virus, lentiviral vectors, baculovirus, reovirus, Newcastle disease virus, alphaviruse and vesicular stomatitis virus vectors.

Retroviruses are single strand, diploid RNA viruses, which enter cells by binding surface envelope proteins, encoded by the env gene. After entering a cell, reverse transcriptase encoded by the pol gene transcribes the viral genome into a double strand DNA copy that can enter the nucleus of dividing cells and integrate randomly into the host genome. Preferably, retroviruses used for viral delivery are manipulated to render them replication deficient by removing their gag, pol and env genes. Thus, infectious but non-replicative retrovirus particles are produced in packaging cell lines that express retrovirus gag, pol and env genes from plasmids lacking a packaging sequence.

The lentiviruses, a subtype of retroviruses, may represent an alternative to retroviruses. Lentiviruses, such as HIV, simian and feline immunodeficiency viruses, can infect non-dividing cells and integrate in the same way as other retroviruses. Replication defective and multiply attenuated lentiviral vectors have been shown to lead to long term expression of various transgenes in the CNS of both rodents and primates (Bensadoun et al. (2000) Exp. Neurol. 164, 15-24; Kordower et al. (2000) Exp. Neurol. 160, 1-16). Lentiviral vectors diffuses 2-3 mm from the injection site which allows the transduction of a significant number of neurones with a sustained gene expression up to at least one year.

Still other viruses are adenoviruses, which comprise double strand DNA viruses. More than 40 adenovirus serotypes in 6 groups (A to F) have been identified. Group C viruses (serotypes Ad2 and Ad5) have been most extensively evaluated as candidates for gene delivery (Zhang (1999) Cancer Gene Ther. 6, 11). Adenoviruses enter cells by binding to the coxsackievirus and adenovirus receptor, which facilitates interaction of viral arginine-glycine-aspartate (RGD) sequences with cellular integrins. After internalisation, the virus escapes from cellular endosomes, partially disassembles and translocates to the nucleus, where viral gene expression begins. Preferably, the adenovirus is incapable of replication. This may be achieved by deleting one or more of the adenovirus genes—such as the early adenovirus genes E1 to E4. This may be extended to remove the whole coding sequence of the adenovirus genome. Such viruses may be used for packaging FLRT-3 genes but must be grown in producer cell lines in the presence of helper viruses that supply all necessary viral gene functions to facilitate the packaging of infectious, replication incompetent adenovirus containing the FLRT-3 gene.

Adeno-associated viruses are single strand DNA viruses that are native human viruses not known to cause any disease. They enter cells via binding to heparan sulfate but require co-infection with a so-called helper virus—such as adenovirus or herpes virus—to replicate. Adeno-associated virus vectors have a number of potential advantages. They infect non-dividing cells and are stably integrated and maintained in the host genome; integration occurs preferentially at a site dependent locus in chromosome 19, decreasing the risk of insertional mutagenesis. However, in adeno-associated virus vectors this characteristic integration is lost due to deletion of rep proteins in an attempt to decrease the risk of the emergence of replication competent adeno-associated viruses.

Herpes simplex viruses are large viruses with a linear double strand DNA genome of approximately 150 kbp that encodes more than 70 viral proteins. These viruses enter cells by binding viral glycoproteins to cell surface heparan sulfate residues. Preferably, herpes simplex viruses are rendered replication defective by inactivating a small number of genes—such as the immediate early genes ICPD, ICP4, 10P22 and ICP27. Since a large number of herpes simplex virus genes can be deleted without affecting the ability to produce viral vectors, large nucleic acid sequences containing multiple genes and their regulatory elements may be packaged within herpes simplex virus vectors.

Pox viruses are double strand DNA viruses that include vaccinia and canarypox or ALVAC. Preferably, the pox virus is a recombinant pox virus containing the FLRT-3 gene.

Methods of delivery by viral techniques are now described in further detail below:

Adenovirus

One method for delivery of nucleic acid constructs comprising FLRT-3 involves the use of an adenovirus expression vector. Although adenovirus vectors are known to have a low capacity for integration into genomic DNA, this feature is counterbalanced by the high efficiency of gene transfer afforded by these vectors.

As used herein, the term “adenovirus expression vector” is meant to include those constructs containing adenovirus sequences sufficient to (a) support packaging of the construct and (b) to ultimately express a construct that has been cloned therein.

The vector comprises a genetically engineered form of adenovirus. Knowledge of the genetic organisation or adenovirus, a 36 kb, linear, double-stranded DNA virus, allows substitution of large pieces of adenoviral DNA with foreign sequences up to 7 kb. In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan (1990) Radiother. Oncol. 19, 197-218). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

Recombinant adenovirus may be generated from homologous recombination between a shuttle vector and a provirus vector. Due to the possible recombination between two proviral vectors, wild-type adenovirus may be generated from this process. Therefore, it is critical to isolate a single clone of virus from an individual plaque and examine its genomic structure.

Generation and propagation of adenovirus vectors, which are replication deficient, depends on a helper cell line that constitutively expresses E1 proteins. Since the E3 region is dispensable from the adenovirus genome (Jones and Shenk (1978) Cell 131, 81-8.), adenovirus vectors with the aid of helper cells, may carry foreign DNA in either the E1, the D3 or both regions. In nature, adenovirus can package approximately 105% of the wild-type genome providing capacity for about 2 extra kb of DNA. Combined with the approximately 5.5 kb of DNA that is replaceable in the E1 and E3 regions, the capacity of the current adenovirus vector is around 7.5 kb, or about 15% of the total length of the vector. More than 80% of the adenovirus viral genome remains in the vector backbone.

Helper cell lines may be derived from human cells—such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells.

Various methods may be used for culturing helper cells and propagating adenovirus. In one format, natural cell aggregates are grown by inoculating individual cells into 1 litre siliconised spinner flasks (Techne, Cambridge, UK) containing 100-200 ml of medium. Following stirring at 40 rpm, the cell viability is estimated with trypan blue. In another format, Fibra-Cel microcarriers (Bibby Sterilin, Stone, UK) (5 g/l) are employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

The adenovirus may be of any of the 42 different known serotypes or subgroups A-F. In some instances, the Adenovirus type 5 of subgroup C is the preferred starting material in order to obtain a conditional replication-defective adenovirus vector. This is because Adenovirus type 5 is a human adenovirus about which a great deal of biochemical and genetic information is known, and it has historically been used for most constructions employing adenovirus as a vector.

Thus, the typical vector is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors or in the E4 region where a helper cell line or helper virus complements the E4 defect.

Adenovirus growth and manipulation is known to those of skill in the art, and exhibits broad host range in vitro and in vivo. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Top et al. (1971) J Infect Dis. 124, 148-54), demonstrating their safety and therapeutic potential as in vivo gene transfer vectors.

Adenovirus vectors have been used in eukaryotic gene expression (Levrero et al. (1991) Gene 101, 195-202; Gomez-Foix et al. (1992) J Biol Chem. 267, 25129-34) and vaccine development (Graham & Prevec (1992) Biotechnol. 20. 363-90). Studies in administering recombinant adenovirus to different tissues include trachea instillation, muscle injection, peripheral intravenous injections and stereotactic inoculation into the brain (Le Gal La Salle et al. (1993) Science 12, 988-90).

Adeno-Associated Virus

Adeno-associated virus (AAV) may also be used in the present invention for the delivery of FLRT-3 as it has a high frequency of integration and may even infect non-dividing cells. Details concerning the generation and use of AAV vectors are described in U.S. Pat. No. 5,139,941 and U.S. Pat. No. 4,797,368.

Recombinant AAV (rAAV) vectors have been used successfully for in vitro and in vivo transduction of marker genes (Kaplitt et al. (1994) Nat Genet 8, 148-54) and genes involved in human diseases.

AAV is a dependent parvovirus in that it requires coinfection with another virus (either adenovirus or a member of the herpes virus family) to undergo a productive infection in cultured cells. In the absence of coinfection with helper virus, the wild type AAV genome integrates through its ends into human chromosome 19 where it resides in a latent state as a provirus. rAAV, however, is not restricted to chromosome 19 for integration unless the AAV Rep protein is also expressed. When a cell carrying an AAV provirus is superinfected with a helper virus, the AAV genome is “rescued” from the chromosome or from a recombinant plasmid, and a normal productive infection is established (Muzyczka (1992) Curr. Top. Microbiol Immunol. 158, 97-129).

Typically, rAAV is made by cotransfecting a plasmid containing the gene of interest flanked by the two AAV terminal repeats and an expression plasmid containing the wild type AAV coding sequences without the terminal repeats. The cells are also infected or transfected with adenovirus or plasmids carrying the adenovirus genes required for AAV helper function. RAAV stocks made in such fashion are contaminated with adenovirus, which must be physically separated from the rAAV particles (for example, by caesium chloride density centrifugation). Alternatively, adenovirus vectors containing the AAV coding regions or cell lines containing the AAV coding regions and some or all of the adenovirus helper genes could be used.

AAV vectors have been successfully used for gene transfer into the brain of rodents and non-human primates (Peel & Kelin (2000) J. Neurosci. Methods 98, 95-104). Owing to their low inflammation property they can be used to infect neurons in regions known to be very reactive such as the spinal cord.

Retrovirus

As mentioned above, the retroviruses are a group of single-stranded RNA viruses characterised by an ability to convert their RNA to double-stranded DNA in infected cells by a process of reverse-transcription. The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from gag contains a signal for packaging of the genome into virions. Two long terminal repeat (LTR) sequences are present at the 5′ and 3′ ends of the viral genome. These contain strong promoter and enhancer sequences and are also required for integration in the host cell genome.

In another aspect, FLRT-3 may be housed within an infective virus that has been engineered to express a specific binding ligand—such as specific FLRT-3 binding ligand. The virus particle will thus bind specifically to the receptors of the target cell and deliver the contents to the cell.

Fragments, Mutants Variants and Homologues

The term “mutant” is used to mean a polypeptide or nucleotide sequence having a sequence which differs from the wild type FLRT-3 sequence by one or more additions, substitutions or deletions. A mutant may arise naturally, or may be created artificially (for example by site-directed mutagenesis). Preferably the mutant has at least 90% sequence identity with the wild type sequence.

The term “variant” is used to mean a naturally occurring polypeptide or nucleotide sequences which differs from the wild-type FLRT-3 sequence. A variant may be found within the same organism or may be found within a different organism. Preferably the variant has at least 90% sequence identity with the wild type sequence.

Here, the term “homologue” means an entity having a certain homology with the wild type FLRT-3 amino acid sequence and the wild type FLRT-3 nucleotide sequence. Here, the term “homology” can be equated with “identity”.

In the present context, an homologous sequence is taken to include an amino acid sequence which may be at least 75, 85 or 90% identical, preferably at least 95 or 98% identical to the FLRT-3 subject sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid # and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term “fragment” indicates that the polypeptide or nucleotide sequence comprises a fraction of the FLRT-3 wild-type sequence. It may comprise one or more large contiguous sections of sequence or a plurality of small sections. The sequence may also comprise other elements of sequence, for example, it may be a fusion protein with another protein. Preferably the sequence comprises at least 50%, 60%, 70%, 80% 90% or 95% of the wild-type sequence.

Assay Method

In one aspect, the present invention relates to a target comprising the amino acid sequence and/or nucleotide sequence of FLRT-3, which may be used in an assay method to identify an agent that modulates the activity of FLRT-3 in the presence or absence of an agent.

Where the candidate agents are proteins e.g. antibodies or peptides, libraries of candidate agents may be screened using phage display techniques. Phage display is a protocol of molecular screening, which utilises recombinant bacteriophage. The technology involves transforming bacteriophage with a gene that encodes the library of candidate agents, such that each phage or phagemid expresses a particular candidate agent. The transformed bacteriophage (which preferably is tethered to a solid support) expresses the appropriate candidate agent and displays it on their phage coat. Specific candidate agents, which are capable of interacting with the target, are identified and enriched by selection strategies based on affinity interaction. The successful candidate agents are then characterised. Phage display has advantages over standard affinity ligand screening technologies. The phage surface displays the candidate agent in a three dimensional configuration, more closely resembling its naturally occurring conformation. This allows for more specific and higher affinity binding for screening purposes.

Another method of screening a library of candidate compounds—e.g. a library of candidate agents—utilises eukaryotic or prokaryotic host cells, which are stably transformed with recombinant DNA molecules expressing the library of candidate agents. Such cells, either in viable or fixed form, can be used for standard binding-partner assays. See also Parce et al. (1989) Science 246:243-247; and Owicki et al. (1990) Proc. Nat'l Acad. Sci. USA 87;4007-4011, which describe sensitive methods to detect cellular responses. Competitive assays are particularly useful, where the cells expressing the library of compounds are incubated with a labelled antibody known to bind to a FLRT-3 polypeptide—such as ¹²⁵I-antibody—and a test sample—such as a candidate compound whose binding affinity to the binding composition is being measured. The bound and free labelled binding partners are then separated to assess the degree of binding. The amount of test sample bound is inversely proportional to the amount of labelled antibody bound. Any one of numerous techniques can be used to separate bound from free binding partners to assess the degree of binding. This separation step could typically involve a procedure such as adhesion to filters followed by washing, adhesion to plastic following by washing, or centrifugation of the cell membranes.

Still another approach is to use solubilised, unpurified or solubilised target protein either extracted from, for example, expressing mammalian cells or from transformed eukaryotic or prokaryotic host cells. This allows for a “molecular” binding assay with the advantages of increased specificity, the ability to automate, and high throughput.

Another technique for candidate agent screening involves an approach which provides high throughput screening for new compounds having suitable binding affinity and is described in detail in WO 84/03564. First, large numbers of different small peptide test compounds are synthesised on a solid substrate, e.g., plastic pins or some other appropriate surface. All the pins are reacted with solubilised target protein and washed. The next step involves detecting bound protein. Detection may be accomplished using for example, a monoclonal antibody to the target protein. Agents which interact specifically with the target protein may thus be identified.

Rational design of candidate agents likely to be able to interact with the target protein may be based upon structural studies of the molecular shapes of the target protein and/or its in vivo binding partners. One means for determining which sites interact with other proteins is a physical structure determination, e.g. X-ray crystallography or two-dimensional NMR techniques. These will provide guidance as to which amino acid residues form molecular contact regions. For a detailed description of protein structural determination, see, e.g., Blundell and Johnson (1976) Protein Crystallography, Academic Press, New York.

The target employed in the assays methods of the present invention may be free in solution, affixed to a solid support, borne on a cell surface, or located intracellularly. It is feasible to immobilise the target to facilitate separation of complexes from uncomplexed forms, as well as to accommodate automation of the assay. Interaction may be accomplished in any vessel suitable for containing the reactants. Examples of such vessels include microtitre plates, test tubes, and microcentrifuge tubes. A fusion protein may be provided which adds a domain that allows the protein to be bound to a matrix. For example, His tagged FLRT-3 can be adsorbed onto Ni-NTA microtitre plates. Following incubation, the plates are washed to remove any unbound label, and the matrix immobilised and radiolabel determined directly, or in the supernatant after the complexes are dissociated. Alternatively, the complexes can be dissociated from the matrix, separated by SDS-PAGE, and the level of FLRT-3 binding protein found in the bead fraction quantified from the gel using standard electrophoretic techniques. Other techniques for immobilising proteins on matrices can also be used in the drug screening assays of the invention. For example, FLRT-3 may be immobilised via conjugation of biotin and streptavidin. Biotinylated FLRT-3 may be prepared from biotin-NHS (N-hydroxy-succinimide) using techniques well known in the art, and immobilized in the wells of streptavidin-coated 96 well plates. Alternatively, antibodies reactive with FLRT-3 but which do not interfere with binding of the protein may be derivatised to the wells of the plate, and FLRT-3 trapped in the wells by antibody conjugation. Preparations of FLRT-3 and an agent are incubated in the FLRT-3-presenting wells of the plate, and the amount of complex trapped in the well can be quantified. Methods for detecting such complexes, may include immunodetection of complexes using antibodies reactive with FLRT-3, or which are reactive with FLRT-3.

Several other types of in vitro assays may be conducted to identify those compounds that modulate the activity of the target protein.

In one assay, purified target protein is immobilised by attachment to the bottom of the wells of a microtiter plate. Radiolabelled target protein, as well as one or more candidate agents are then be added either one at a time or simultaneously to the wells. After incubation, the wells are washed and counted using a scintillation counter for radioactivity to determine the degree of target protein binding in the presence of the candidate agents. Typically, binding will be tested over a range of concentrations and a series of control “wells” lacking one or more elements of the assays are used for accuracy in evaluating the results.

Several means are available to “mark” the target protein. By way of example, the target protein can be radiolabelled using, for example, ¹²⁵I or ³⁵S. Alternatively, a fusion protein may be prepared comprising the target protein fused to the coding sequence of a peptide such as an epitope tag e.g. the c-myc epitope. The c-myc fusion protein can be readily detected with commercially available antibodies directed against myc.

Another type of in vitro assay that is useful for identifying a molecule that modulates target protein activity is the Biacore assay system (Pharmacia, Piscataway, N.J.) using a surface plasmon resonance detector system and following the manufacturer's protocol. This assay essentially involves covalent binding of the target protein to a dextran-coated sensor chip which is located in a detector. The candidate agent(s) can be injected into the chamber containing the sensor chip either simultaneously or sequentially, and the amount of binding can be assessed based on the change in molecular mass which is physically associated with the dextran-coated side of the sensor chip; the change in molecular mass can be measured by the detector system.

In some cases, it may be desirable to evaluate two or more candidate agents together for use in modulating the activity of the target. In these cases, assays can be readily modified by adding such additional candidate agent(s) either simultaneously with, or subsequently to, the first candidate agent.

The method of the present invention may also be a screen, whereby a number of candidate agents are tested for modulating the activity of the target.

This invention also contemplates the use of competitive drug screening assays in which neutralising antibodies capable of binding a target specifically compete with a candidate agent for binding to a target.

It is expected that the assay methods of the present invention will be suitable for both small and large-scale screening of candidate agents as well as in quantitative assays.

Preferably, the assay method of the present invention, comprises the step of determining if the agent modulates the ability of FLRT-3 to regenerate neurons.

Determining the Activity of FLRT-3

The activity—such as the expression—of FLRT-3 may be determined using various methods known in the art, for example, Northern blotting, in situ hybridisation, nuclease protection assay and RT-PCR.

Northern blotting is a technique for size fractionating RNA in a gel, followed by transfer and immobilisation on a solid support, for example, a membrane in such a manner that the relative positions of the RNA molecules are maintained. The resulting Northern blot is then hybridised with a labelled probe complementary to the mRNA of interest. Signal generated from detection of the probe can be used to determine the size and abundance of the target RNA.

Northern blotting analysis may be performed using total RNA and a radioactively labelled FLRT-3 fragment.

The steps involved in Northern analysis include: RNA isolation (total or poly(A) RNA), probe generation, denaturing agarose gel electrophoresis, transfer to solid support and immobilization, prehybridisation and hybridization with probe, washing, detection and finally stripping and reprobing (optional). Kits for practising Northern blotting are commercially available such as the NorthernMax Kit (Ambion).

In situ hybridisation (ISH) may also be used. Various probes may be used in this method—such as RNA probes, which may be generated and labelled by in vitro transcription procedures or synthetic oligonucleotide probes which may be prepared using various methods known in the art e.g. by automated chemical synthesis. Oligonucleotide probes are labelled during synthesis or by addition of reporter molecules at the 5′ or 3′ end after synthesis. Probes may be labelled in various ways, using radioactive labels e.g. ³²P, or non-radicoactibe labels e.g. DIG. The ISH method comprises a number of steps as briefly outlined below:

i. Prehybridisation—Samples e.g. sections of tissues, to be hybridised are incubated with phosphate buffered saline (PBS), washed and then permeabilised with buffer containing either RNase-free Proteinase K or RNase-free Proteinase K. Samples are post-fixed with PBS containing para-formaldehyde and washed with PBS. Samples are acetylated with TEA buffer containing acetic anhydride and incubated with prehybridisation buffer.

ii. In situ hybridisation—A hybridisation buffer is prepared and slides are dipped briefly in distilled water. Prehybridisation buffer is drained from the slides and overlayed with the hybridisation buffer containing labelled probe. Samples are covered and incubated overnight in a humid chamber.

iii Posthybridisation—Coverslips are removed and samples washed. Unbound RNA probe is removed and the samples again washed.

iv Immunological detection—Samples are washed with buffer and covered with blocking solution, followed by incubation in a humid chamber with a suitable dilution of labelled antibody—such as sheep anti-DIG-alkaline phosphatase. Samples are again washed and a colour solution prepared containing, for example, nitroblue tetrazolium solution, 5-bromo-4-chloro-3-indolyl-phosphate and levamisole. Each sample is covered with the colour solution and slides are incubated in a humid chamber in the dark. When colour development is optimal, the color reaction is stopped and slides are dipped briefly in distilled water. Samples are counterstained and washed.

Additional information concerning in situ hybridisation is available in Komminoth (1992) Diagn. Mol. Pathol. 1, 142-150, Komminoth et al. (1992) Histochemistry 98, 217-228 and Sambrook et al. Molecular Cloning, A Laboratory Manual (1989).

Still another method for determining the activity of FLRT-3 is the Nuclease Protection Assay, which may be used to detect, and quantify specific FLRT-3 mRNA species. A radioactive or non-radioactively labelled single-stranded DNA (or RNA) probe is hybridised to the FLRT-3 target mRNA. S1 nuclease is then used to digest all single-stranded RNA and non-duplexed probe. Under optimised conditions, S1 nuclease is highly specific for degrading single-stranded DNA and RNA while exhibiting low activity directed toward DNA or RNA in a DNA:RNA (or RNA:RNA) duplex. The remaining double-stranded hybrid is recovered by ethanol precipitation, separated by gel electrophoresis and visualised by autoradiography. Kits for performing the Nuclease Protection Assay are commercially available—such as the S1-Assay (Ambion).

Yet another method is RT-PCR which is both a sensitive and versatile method that can be used to determine the activity of FLRT-3. Since RNA cannot serve as a template for PCR, reverse transcription is combined with PCR to make complementary DNA (cDNA) suitable for PCR. This combination of both technologies is referred to as RT-PCR. The technique can be used to, amongst other things, determine the presence or absence of a transcript and to estimate expression levels. Various types of primers may be used for reverse transcription—such as oligo (dT)12-18 which binds to the poly (A) tail at the 3′ end of mammalian RNA; Random hexanucleotides which bind to mRNA at any complementary site and may be better for overcoming difficulties caused by template secondary structure; and specific oligonucleotide primers can be used to selectively prime the FLRT-3 RNA sequence of interest. Selection of an appropriate primer for reverse transcription is dependent upon the size and secondary structure of the FLRT-3 mRNA.

Preferably, sequence specific primers that anneal only to defined sequences in particular RNAs—such as FLRT-3 mRNAs—rather than to the entire RNA population in the sample (e.g., random hexamers or oligo(dT)) are used.

RT-PCR may be one-tube or two-tube. In one-tube RT-PCR, both reverse transcription and PCR are performed in the same tube. A thermostable DNA polymerase—such as Taq DNA polymerase—and all necessary primers and reagents are added during the reaction set-up. cDNA is synthesised by reverse transcription at 37° C. while the DNA polymerase in the reaction mix has little activity. After allowing sufficient time for reverse transcription, the temperature is raised, inactivating the reverse transcriptase and allowing the DNA polymerase to amplify the cDNA. In two-tube RT-PCR, cDNA is first synthesised by reverse transcription. An aliquot of the finished reverse-transcription reaction is then used for PCR. Kits for performing RT-PCR are commercially available—such as the Polymerase One-Step RT-PCR System (Roche Molecular Biochemicals), Titan One-Tube RT-PCR Kit (Roche Molecular Biochemicals) and OneStep RT-PCR Kit (Qiagen).

It will be understood by a skilled person that other methods can be used to determine the activity of FLRT-3.

Modulating

As used herein, the term “modulates” refers to preventing, suppressing, alleviating, restorating, elevating or otherwise affecting the activity—such as the expression—of FLRT-3.

Agent

As used herein, the term “agent” may be a single entity or it may be a combination of entities. Preferably, the agent modulates the activity of FLRT-3.

The agent may modulate the activity of FLRT-3 to typically increase the regenerative potential of the neuron. Thus, the agent may be an antagonist or an agonist of FLRT-3. Preferably, the agent is an agonist of FLRT-3.

The agent may modulate the interaction of FLRT-3 with another molecule—such as a ligand of FLRT-3—which may be accomplished by an agent that typically promotes the interaction of FLRT-3 with another molecule. Thus, the agent may be an antagonist or an agonist of another molecule—such as a ligand of FLRT-3—which may increase the regenerative potential of the neuron. Preferably, the agent is an agonist of another molecule—such as a ligand of FLRT-3.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may even be an antibody.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the agent will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent comprises at least the one of said cyclic groups linked to another hydrocarbyl group.

The agent may contain halo groups, for example, fluoro, chloro, bromo or iodo groups.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, (1977) J. Pharm. Sci. 66, 1-19.

The agent of the present invention may be capable of displaying other therapeutic properties.

The agent may be used in combination with one or more other pharmaceutically active agents.

If combinations of active agents are administered, then they may be administered simultaneously, separately or sequentially.

Antagonist

As used herein, the term “antagonist” means any agent that reduces the action—such as the expression—of another agent or target—such as FLRT-3.

Agonist

As used herein the term “agonist” means any agent that enhances the action—such as the expression—of or activates another agent or target—such as FLRT-3. The term agonist may include a ligand that binds to one or more receptors—such as a FLRT-3 receptor—and thereby alters, typically increases, the proportion of them that are in an active form, resulting in a biological response—such as the regeneration of neurons.

Stereo and Geometric Isomers

Agents may exist as stereoisomers and/or geometric isomers—e.g. the agents may possess one or more asymmetric and/or geometric centres and so may exist in two or more stereoisomeric and/or geometric forms. The present invention contemplates the use of all the individual stereoisomers and geometric isomers of those agents, and mixtures thereof.

Pharmaceutical Salt

Agents may be administered in the form of a pharmaceutically acceptable salt.

Pharmaceutically-acceptable salts are well known to those skilled in the art, and for example include those mentioned by Berge et al, (1977) J. Pharm. Sci., 66, 1-19. Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of the agent and the desired acid or base, as appropriate. The salt may precipitate from solution and be collected by filtration or may be recovered by evaporation of the solvent.

The agent may exisit in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The agent may also include all suitable isotopic variations of the agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, 18O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof of this invention can generally be prepared by conventional procedures using appropriate isotopic variations of suitable reagents.

It will be appreciated by those skilled in the art that the agent may be derived from a prodrug. Examples of prodrugs include entities that have certain protected group(s) and which may not possess pharmacological activity as such, but may, in certain instances, be administered (such as orally or parenterally) and thereafter metabolised in the body to form the agent which is pharmacologically active.

It will be further appreciated that certain moieties known as “pro-moieties”, for example as described in “Design of Prodrugs” by H. Bundgaard, Elsevier, 1985 (the disclosured of which is hereby incorporated by reference), may be placed on appropriate functionalities of the agents. Such prodrugs are also included within the scope of the invention.

Pharmaceutically Active Salt

The agent may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Chemical Synthesis Methods

FLRT-3 or agents of the present invention may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts may be separated and purified by conventional methods.

Separation of diastereomers may be achieved by conventional techniques, e.g. by fractional crystallisation, chromatography or H.P.L.C. of a stereoisomeric mixture of a compound of formula (I) or a suitable salt or derivative thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

FLRT-3, agents or variants, homologues, derivatives, fragments or mimetics thereof may be produced using chemical methods to synthesise the FLRT-3 or the agent in whole or in part. For example, a FLRT-3 peptide or an agent that is a peptide can be synthesised by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Synthesis of peptide inhibitor agents (or variants, homologues, derivatives, fragments or mimetics thereof) can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent, may be altered during direct synthesis and/or combined using chemical methods with a sequence from other subunits, or any part thereof, to produce a variant agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

The agent may be a chemically modified agent.

The chemical modification of an agent may either enhance or reduce hydrogen bonding interaction, charge interaction, hydrophobic interaction, Van Der Waals interaction or dipole interaction between the agent and the target.

In one aspect, the agent may act as a model (for example, a template) for the development of other compounds.

Pharmaceutical Compositions

The components may be administered alone but will generally be administered as a pharmaceutical composition—e.g. when the components are in admixture with a suitable pharmaceutical excipient, diluent or carrier selected with regard to the intended route of administration and standard pharmaceutical practice.

For example, the components can be administered in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders—such as polyvinylpyrrolidone, hydroxypropylmethylcellulose (HPMC), hydroxypropylcellulose (HPC), sucrose, gelatin and acacia. Additionally, lubricating agents—such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intraventricular, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Preferably, the routes of administration (delivery) include administration to the CNS. More preferably, the routes of administration achieves significant CNS target site concentrations while limiting systemic exposure and distribution to peripheral sites of action to lessen unwanted pleiotropic effects and toxicity.

Local introduction of FLRT-3 and agents etc. into the CNS intraparenchymally by direct injection/infusion or by implantation of delivery vectors—such as polymer matrices or genetically modified cells—may be used. Delivery of FLRT-3 and agents etc into the cerebrospinal fluid (CSF) following intracerebroventricular or intrathecal administration is less invasive and may allow access to a much wider area of the CNS through CSF circulation pathways. Intranasal administration may also offer some degree of CNS targeting with minimal invasiveness.

For a review of the various therapies available for CNS repair, see Aebischer & Ridet (2001) Trends Neurosci. 24, 533-540 and Berry et al. (2001) Current Opinion in Mol. Ther. 3, 338-349.

Pharmaceutical compositions of the present invention may comprise a therapeutically effective amount of FLRT-3, one or more agents or combinations thereof.

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The agents may be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If the agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Formulation

The component(s) may be formulated into a pharmaceutical composition, such as by mixing with one or more of a suitable carrier, diluent or excipient, by using techniques that are known in the art.

The invention will now be further described by way of Examples, which are meant to serve to assist one of ordinary skill in the art in carrying out the invention and are not intended in any way to limit the scope of the invention.

Amino Acid Sequence

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

In one aspect, the present invention provides an amino acid sequence that is capable of acting as a target in an assay for the identification of one or more agents and/or derivatives thereof.

Preferably, the target is FLRT-3 comprising the sequences set forth in Seq ID No.2. FLRT-3 may be in a substantially pure form, in which case it will generally comprise FLRT-3 in a preparation in which more than 90%, e.g. 95%, 98% or 99% of FLRT-3 in the preparation is a peptide obtainable from the expression of SEQ ID No. 1 or variants, homologues, derivatives or fragments thereof or a peptide comprising the amino acid sequence shown as SEQ ID NO. 2 or variants, homologues, derivatives or fragments thereof.

It will be understood that FLRT-3 may be mixed with carriers or diluents which will not interfere with the intended purpose of the molecule and which will still be regarded as substantially isolated.

Nucleotide Sequence

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form for this aspect.

In one aspect, the present invention provides a nucleotide sequence that is capable of acting as a target in an assay for the identification of one or more agents and/or derivatives thereof.

Preferably, the target is FLRT-3 comprising the sequences set forth in Seq ID No. 1.

It will be understood by a skilled person that numerous different nucleotide sequences can encode the same target as a result of the degeneracy of the genetic code. In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to nucleotide sequences include any substitution of, variation of, modification of, replacement of, deletion of or addition of one (or more) nucleic acids from or to the sequence providing the resultant nucleotide sequence encodes a functional target according to the present invention (or even an agent according to the present invention if said agent comprises a nucleotide sequence or an amino acid sequence).

Hybridisation

The term “hybridisation” as used herein shall include “the process by which a strand of nucleic acid joins with a complementary strand through base pairing” as well as the process of amplification as carried out in, for example, PCR or RT-PCR.

Nucleotide sequences capable of selectively hybridising to the nucleotide sequences presented herein—such as FLRT-3—or to their complement, will be generally at least 75%, preferably at least 85 or 90% and more preferably at least 95% or 98% homologous to the corresponding complementary nucleotide sequences presented herein over a region of at least 20, preferably at least 25 or 30, for instance at least 40, 60 or 100 or more contiguous nucleotides.

The term “selectively hybridisable” means that the nucleotide sequence, when used as a probe, is used under conditions where a target nucleotide sequence is found to hybridise to the probe at a level significantly above background. The background hybridisation may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with ³²P.

Hybridisation conditions are based on the melting temperature (Tm) of the nucleic acid binding complex, as taught in Berger and Kimmel (1987, Guide to Molecular Cloning Techniques, Methods in Enzymology, Vol. 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridisation can be used to identify or detect identical nucleotide sequences while an intermediate (or low) stringency hybridisation can be used to identify or detect similar or related polynucleotide sequences.

Nucleotide sequences which are not 100% homologous to the sequences described herein but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries or genomic DNA libraries from other animal species, and probing such libraries with probes comprising all or part of the nucleotide sequence set out in herein under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set forth in Seq. ID No 1. This may be useful where for example silent codon changes are required in sequences to optimise codon preferences for a particular host cell in which the nucleotide sequences are being expressed. Other sequence changes may be desired in order to introduce restriction enzyme recognition sites, or to alter the activity of the protein encoded by the nucleotide sequences.

The nucleotide sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer nucleotide sequences will generally be produced using recombinant means, for example using PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express the target sequences. As will be understood by those of skill in the art, for certain expression systems, it may be advantageous to produce the target sequences with non-naturally occurring codons. Codons preferred by a particular prokaryotic or eukaryotic host (Murray E et al (1989) Nuc Acids Res 17:477-508) can be selected, for example, to increase the rate of the target expression or to produce recombinant RNA transcripts having desirable properties, such as a longer half-life, than transcripts produced from naturally occurring sequence.

Vector

Aspects of the present invention relate to a vector comprising a nucleotide sequence—such as a nucleotide sequence encoding FLRT-3 or an agent—administered to a subject.

Preferably, FLRT-3 or the agent is prepared and/or delivered to a target site using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising nucleotide sequences and/or expressing the proteins encoded by the nucleotide sequences. Examples of vectors used in recombinant DNA techniques include but are not limited to plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

Regulatory Sequences

In some applications, nucleotide sequences are operably linked to a regulatory sequence which is capable of providing for the expression of the nucleotide sequence, such as by a chosen host cell. By way of example, a vector comprising the FLRT-3 nucleotide sequence is operably linked to such a regulatory sequence i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals.

The term “promoter” is used in the normal sense of the art, e.g. an RNA polymerase binding site.

Enhanced expression of the FLRT-3 nucleotide sequence may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of FLRT-3. In eukaryotes, polyadenylation sequences may be operably connected to the FLRT-3 nucleotide sequence.

Preferably, the FLRT-3 nucleotide sequence is operably linked to at least a promoter.

Aside from the promoter native to the gene encoding the FLRT-3 nucleotide sequence, other promoters may be used to direct expression of the FLRT-3 polypeptide. The promoter may be selected for its efficiency in directing the expression of the FLRT-3 nucleotide sequence in the desired expression host.

In another embodiment, a constitutive promoter may be selected to direct the expression of the FLRT-3 nucleotide sequence of the present invention. Such an expression construct may provide additional advantages since it circumvents the need to culture the expression hosts on a medium containing an inducing substrate.

Examples of suitable promoters for directing the transcription of the FLRT-3 nucleotide sequence in a bacterial host include the promoter of the lac operon of E. coli, the Streptomyces coelicolor agarase gene dagA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase gene (amyQ), the promoters of the Bacillus subtilis xylA and xylB genes and a promoter derived from a Lactococcus sp.-derived promoter including the P170 promoter. When the FLRT-3 nucleotide sequence is expressed in a bacterial species such as E. coli, a suitable promoter can be selected, for example, from a bacteriophage promoter including a T7 promoter and a phage lambda promoter.

For transcription in a fungal species, examples of useful promoters are those derived from the genes encoding the, Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, Aspergillus oryzae alkaline protease, Aspergillus oryzae triose phosphate isomerase or Aspergillus nidulans acetamidase.

Examples of strong constitutive and/or inducible promoters which are preferred for use in fungal expression hosts are those which are obtainable from the fungal genes for xylanase (xlnA), phytase, ATP-synthetase, subunit 9 (oliC), triose phosphate isomerase (tpi), alcohol dehydrogenase (AdhA), α-amylase (amy), amyloglucosidase (AG—from the glaA gene), acetamidase (amdS) and glyceraldehyde-3-phosphate dehydrogenase (gpd) promoters. Other examples of useful promoters for transcription in a fungal host are those derived from the gene encoding A. oryzae TAKA amylase, the TPI (triose phosphate isomerase) promoter from S. cerevisiae (Alber et al (1982) J. Mol. Appl. Genet. 1, p419-434), Rhizomucor miehei aspartic proteinase, A. niger neutral α-amylase, A. niger acid stable α-amylase, A. niger glucoamylase, Rhizomucor miehei lipase, A. oryzae alkaline protease, A oryzae triose phosphate isomerase or A. nidulans acetamidase.

Examples of suitable promoters for the expression in a yeast species include but are not limited to the Gal 1 and Gal 10 promoters of Saccharomyces cerevisiae and the Pichia pastoris AOX1 or AOX2 promoters.

Examples of strong bacterial promoters are the α-amylase and SP02 promoters as well as promoters from extra-cellular protease genes.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the FLRT-3 nucleotide sequence. For example, suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat (1987) Gene 217, 217-225 and Dawson (1993) Plant Mol. Biol. 23: 97).

Constructs

The term “construct”—which is synonymous with terms such as “conjugate”, “cassette” and “hybrid”—includes a FLRT-3 nucleotide sequence directly or indirectly attached to a promoter. An example of an indirect attachment is the provision of a suitable spacer group such as an intron sequence, such as the Sh1-intron or the ADH intron, intermediate the promoter and the FLRT-3 nucleotide sequence. The same is true for the term “fused” in relation to the present invention, which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct in, for example, a bacterium. Various markers exist which may be used, such as for example those encoding mannose-6-phosphate isomerase (especially for plants) or those markers that provide for antibiotic resistance—e.g. resistance to G418, hygromycin, bleomycin, kanamycin and gentamycin.

For some applications, preferably the construct of the present invention comprises at least the FLRT-3 nucleotide sequence operably linked to a promoter.

Expression Vector

Preferably, nucleotide sequences—such as nucleotide sequences encoding agents of the present invention or FLRT-3—are inserted into a vector that is operably linked to a control sequence that is capable of providing for the expression of the coding sequence by the host cell.

Nucleotide sequences produced by a host recombinant cell may be secreted or may be contained intracellularly depending on the sequence and/or the vector used. As will be understood by those of skill in the art, expression vectors can be designed with signal sequences which direct secretion of the nucleotide sequence through a particular prokaryotic or eukaryotic cell membrane.

Fusion Proteins

FLRT-3 or an agent—such as a FLRT-3 receptor agonist—may be expressed as a fusion protein to aid extraction and purification and/or delivery of the agent of the present invention or the FLRT-3 receptor target to an individual and/or to facilitate the development of a screen for agents. Examples of fusion protein partners include glutathione-S-transferase (GST), 6xHis, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

Host Cells

A wide variety of host cells can be employed for expression of the nucleotide sequences encoding the agent—such as an agent of the present invention—or a FLRT-3 target of the present invention. These cells may be both prokaryotic and eukaryotic host cells. Suitable host cells include bacteria such as E. coli, yeast, filamentous fungi, insect cells, mammalian cells, typically immortalised, e.g., mouse, rat, CHO, human and monkey cell lines and derivatives thereof.

Examples of suitable expression hosts within the scope of the present invention are fungi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

Preferred host cells are able to process the expression products to produce an appropriate mature polypeptide. Examples of processing includes but is not limited to glycosylation, ubiquitination, disulfide bond formation and general post-translational modification.

Antibodies

Aspects of the present invention relate to antibodies. By way of example, such antibodies may combine with FLRT-3 or they may comprise agents that modulate the activity—such as the expression—of FLRT-3.

Antibodies may be produced by standard techniques, such as by immunisation with the substance of the invention or by using a phage display library.

For the purposes of this invention, the term “antibody” includes but is not limited to, polyclonal, monoclonal, chimeric, single chain, Fab fragments, fragments produced by a Fab expression library, as well as mimetics thereof. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralising antibodies, i.e., those which inhibit biological activity of the substance polypeptides, are especially preferred for therapeutics.

Humanised antibodies may be obtained using other methods well known in the art (for example as described in U.S. Pat. No. 239,400).

If polyclonal antibodies are desired, a selected mammal (e.g., mouse, rabbit, goat, horse, etc.) is immunised with an immunogenic polypeptide bearing epitope(s) obtainable from, for example, FLRT-3 or agents that modulate FLRT-3. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope contains antibodies to other antigens, the polyclonal antibodies can be purified by immunoaffinity chromatography. Techniques for producing and processing polyclonal antisera are known in the art.

Monoclonal antibodies directed against epitopes can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulphide bridges of the F(ab′)₂ fragments. Fab expression libraries may also be constructed to allow rapid and easy identification of monoclonal Fab fragments with the desired specificity (Huse W D et al (1989) Science 256:1275-1281).

Reporters

A wide variety of reporters may be used in the assay methods of the present invention with preferred reporters providing conveniently detectable signals (e.g. by spectroscopy). By way of example, a reporter gene may encode an enzyme which catalyses a reaction, which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts, which are then identified when bound to oligonucleotide probes.

For example, the production of the reporter molecule may be measured by the enzymatic activity of the reporter gene product, such as β-galactosidase.

A variety of protocols for use in the assay methods of the present invention are available—such as by using either polyclonal or monoclonal antibodies specific for a protein to be detected—such as FLRT-3. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on polypeptides is preferred, but a competitive binding assay may be employed. These and other assays are described, among other places, in Hampton R et al (1990, Serological Methods, A Laboratory Manual, APS Press, St Paul Minn.) and Maddox D E et al. (1983) J. Exp. Med. 158:1211).

A wide variety of labels and conjugation techniques are known by those skilled in the art and can be used in various nucleic and amino acid assays. Means for producing labelled hybridisation or PCR and RT-PCR probes for detecting the target polynucleotide sequences include oligolabelling, nick translation, end-labelling or PCR amplification using a labelled nucleotide. Alternatively, the coding sequence, or any portion of it, may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; US-A-3,996,345; U.S. Pat. No. 4,277,437; U.S. Pat. No. 4,275,149 and U.S. Pat. No. 4,366,241. Also, recombinant immunoglobulins may be produced as shown in U.S. Pat. No. 4,816,567.

Additional methods to quantify the expression of a particular molecule include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Host cells which contain the coding sequence of interest and express the coding regions of, for example, FLRT-3 may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

Organism

The term “organism” in relation to the present invention includes any organism that could comprise FLRT-3 and/or agents that modulate FLRT-3. Examples of organisms may include mammals, fungi, yeast or plants.

Preferably, the organism is a mammal. More preferably, the organism is a human.

Transformation

As indicated earlier, the host organism can be a prokaryotic or a eukaryotic organism.

5 Examples of suitable prokaryotic hosts include E. coli and Bacillus subtilis. Teachings on the transformation of prokaryotic hosts are well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc. Examples of suitable eukaryotic hosts include mammalian cells.

If a prokaryotic host is used then the nucleotide sequence—such as the FLRT-3 nucleotide sequence—may need to be suitably modified before transformation—such as by removal of introns.

In another embodiment the transformed organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

Thus, the present invention also relates to the transformation of a host cell with a nucleotide sequence—such as FLRT-3 or an agent that modulates FLRT-3. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain, which will facilitate purification of soluble proteins (Kroll D J et al (1993) DNA Cell Biol 12:441-53) e.g. 6-His or Glutathione-S-transferase.

Transfection

Vectors comprising for example, the FLRT-3 nucleotide sequence or an agent that modulates FLRT-3 may be introduced into host cells, for example, mammalian cells, using a variety of methods.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotech. (1996) 14, 556), multivalent cations such as spermine, cationic lipids or polylysine, 1, 2,-bis (oleoyloxy)-3-(trimethylammonio) propane (DOTAP)-cholesterol complexes (Wolff and Trubetskoy 1998 Nature Biotechnology 16: 421) and combinations thereof.

Uptake of nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Such methods are described in many standard laboratory manuals—such as Sambrook et al., Molecular Cloning: A Laboratory Manual, 2d ed. (1989) Cold Spring Harbor Laboratory Press, Cold Spring Harbor, N.Y.

Construction of Non-Human Animals

In a further aspect, the present invention provides a non-human animal having a silenced or eliminated or removed FLRT-3 and/or a silenced or eliminated or removed nucleotide sequence coding FLRT-3. Particular examples of non-human animals include non-human primates, cats, dogs, ungulates such as cows, goats, pigs, sheep and horses and rodents such as mice, rats, gerbils and hamsters.

Techniques for producing non-human animals (also known as transgenic animals) are well known in the art. A useful general textbook on this subject is Houdebine, Transgenic animals—Generation and Use (Harwood Academic, 1997)—which provides an extensive review of the techniques used to generate transgenic animals from fish to mice and cows.

Advances in technologies for embryo micromanipulation now permit introduction of heterologous DNA—for example, FLRT-3 DNA—into, for example, fertilised mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. See reviews of standard laboratory procedures for microinjection of heterologous DNAs into mammalian fertilized ova, including Hogan et al., Manipulating the Mouse Embryo, (Cold Spring Harbor Press 1986); Krimpenfort et al., Bio/Technology 9:844 (1991); Palmiter et al., Cell, 41: 343 (1985); Kraemer et al., Genetic manipulation of the Mammalian Embryo, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated herein by reference.

Another method used to produce a transgenic animals involves microinjecting a nucleic acid into pro-nuclear stage eggs by standard methods. Injected eggs are then cultured before transfer into the oviducts of pseudopregnant recipients. Transgenic animals may also be produced by nuclear transfer technology as described in Schnieke, A. E. et al., 1997, Science, 278: 2130 and Cibelli, J. B. et al., 1998, Science, 280: 1256. Using this method, fibroblasts from donor animals are stably transfected with a plasmid incorporating the coding sequences for a binding domain or binding partner of interest under the control of regulatory. Stable transfectants are then fused to enucleated oocytes, cultured and transferred into female recipients.

Analysis of non-human animals which may contain transgenic sequences would typically be performed by either PCR or Southern blot analysis following standard methods.

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The invention will now be further described by way of Examples, which are meant to serve to assist one of ordinary skill in the art in carrying out the invention and are not intended in any way to limit the scope of the invention.

### EXAMPLES

### Methods

Differential Display

Differential display (DD) of RNA is carried out on total RNA from rat DRG after 3 day sciatic crush (Livesay et al. (1997) Nature 390, 614-618) and the up-regulated FLRT-3 fragment isolated.

A rat hippocampal cDNA library was screened with the DD fragment random primed using ³²PdATP and a 2.8 kb FLRT-3 clone (including the full open reading frame) is obtained.

Northern Blot Analysis

Northern blot analysis is carried out using a random primed using ³²PdATP FLRT-3 fragment. Total RNA is prepared from rat DRG and spinal cord (untreated and after timecourses of sciatic cut and peripheral inflammation).

In-Situ Hybridisation

Radio-isotopic in-situ hybridisation (ISH) is carried out (Wisden & Morris Ed. (1994) In Situ Hybridiastion Protocols for the Brain, Academic Press Limited) for FLRT-3 on cryosectioned adult rat DRG and spinal cord (untreated and after sciatic cut and crush), adult rat brain and E17 rat.

Wholemount ISH is carried out using a digoxygenin (DIG)-labelled FLRT-3 riboprobe on E12rat.

Cellular Localisation

Cellular localisation of FLRT-3 is ascertained in COS-7 cells by transfection with a Myc-tagged FLRT-3 construct and subsequent immunostaining for Myc and FLRT-3.

Antisense Knockdown

Lumbar DRG from two 250-300 g adult male rats (sacrificed by CO₂ inhalation) are dissected out into pre-chilled MEM medium with HEPES. The ganglia are then placed in 3 ml of 3.33 mg/ml collagenase type IV in Hams F14 medium (supplemented with L-glutamine and penicillin-streptomycin) and incubated for 2.5 hours at 37° C. in 5% CO₂. The ganglia are washed free of collagenase in 10 ml of F14 medium and pelleted by centrifugation at 1000 rpm for 1 minute. The F14 medium is decanted off and the ganglia resuspended in 2 ml of F14 medium with 10% heat-inactivated foetal bovine serum. The ganglia are then dissociated into single cells by trituration through a fire-polished glass Pasteur pipette. The neuronal cells are separated from cellular debris by centrifugation (850 rpm, 4 minutes) through a column of 15% BSA in F14 medium. The supernatant is removed and the cells resuspended in 2 ml of Neurobasal medium (with L-glutamine, penicillin-streptomycin, and B27 serum supplement). 100 μl of the cell suspension are then plated in the centre of 22 mm diameter glass coverslips (in 35 mm tissue culture dishes) coated with a poly-DL-ornithine-laminin substratum. The cells are left to adhere to the coverslips for 1 hour at 37° C. in 5% CO₂, after which a further 1 ml of Neurobasal medium is added. The cells are then incubated for 24 hours at 37° C. in 5% CO₂. The Neurobasal medium is removed and sense, antisense, and scrambled FITC-conjugated oligonucleotides are applied to the cells at a concentration of 0.5 μM in pluronic gel. 2 ml of Neurobasal medium are reapplied. The cells are incubated for a further 24 hours, fixed in 4% paraformaldehyde, and immunostained for the pan-neuronal marker PGP9.5 using the DAB reaction.

Overexpression

Cultures are prepared as for the antisense oligonucleotide study using lumbar DRG from one adult male rat. Neuronal cells are left in culture for 24 hours prior to the application of FLRT-3—or control GFP-expressing partially disabled Herpes Simplex virus. Neurobasal medium is removed from the cultures and 10 μl of virus added in 1 ml of F14 medium. The cultures are incubated at 37° C./5% CO₂ for 1 hour, after which time the virus containing medium is removed and replaced with 1 ml of Neurobasal medium. The cells are then incubated for a further 24 hours, fixed in 4% paraformaldehyde, and immunostained for PGP9.5 using the DAB reaction. Neurite outgrowth measurements are carried out using Leica Q Win software.

### Example 1

### Structure of FLRT-3

We have used differential display of mRNA and cDNA library screening to isolate a gene encoding a protein, previously identified as FLRT-3 in human muscle samples (FIG. 1; Lacy S E et al. (1999) Genomics 15;62:417-26). It is demonstrated herein that FLRT-3 is up-regulated in adult rat DRG 3 days after sciatic nerve crush.

Structure prediction analysis (FIG. 7) suggests that FLRT-3 is a single pass transmembrane protein comprising a 530 amino acid N-terminal extracellular domain, a 20 amino acid transmembrane domain, and a 99 amino acid C-terminal intracellular domain. Much of the extracellular face consists of a 320 amino acid leucine rich region, containing 10 leucine rich repeats, which is flanked by cysteine rich regions. A fibronectin type III domain follows this. The intracellular region contains no recognisable motifs. This indicates that FLRT-3 may be a cell surface receptor or cell adhesion molecule. It is similar in some respects to NOGO A receptor but there is little homology at the protein or nucleotide level.

### Example 2

### Expression of FLRT-3

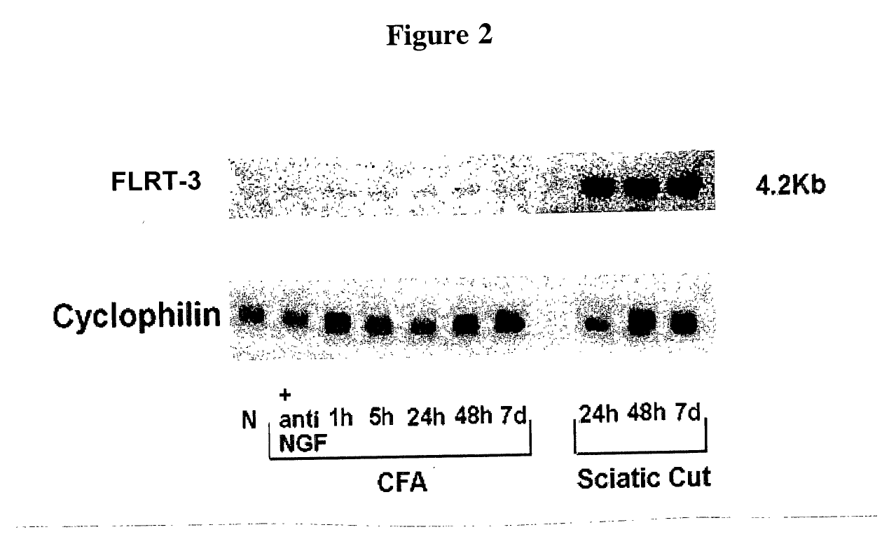

As shown in FIG. 2, FLRT-3 mRNA is up-regulated in adult rat lumbar DRG after sciatic cut. Up-regulation is consistent between 24 hours and 7 days post cut. Peripheral inflammation induced by injection of complete Freund's adjuvant (CFA) into the rat hind paw has no effect on FLRT-3 expression. The northern membrane reprobed with constitutively expressed cyclophilin shows equivalent loading of total RNA. (N, naive; NGF, nerve growth factor; h, hours; d, day.)

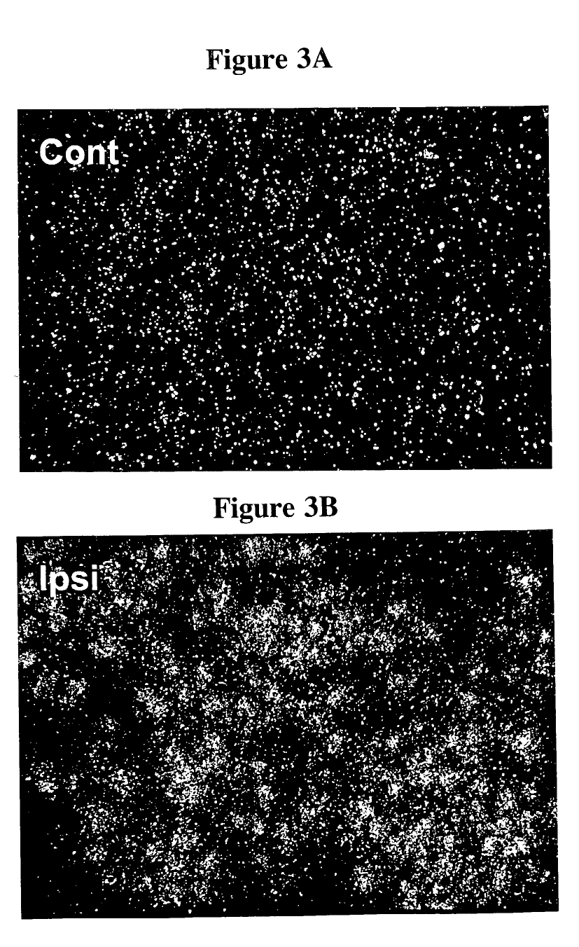

Using radio-isotopic ISH, FIG. 3 demonstrates that FLRT-3 mRNA is up-regulated in most neurons of ipsilateral (Ipsi) adult rat lumbar DRG 3 days after sciatic cut. The DRG from the contralateral (uncut) (Cont) side is shown for comparison.

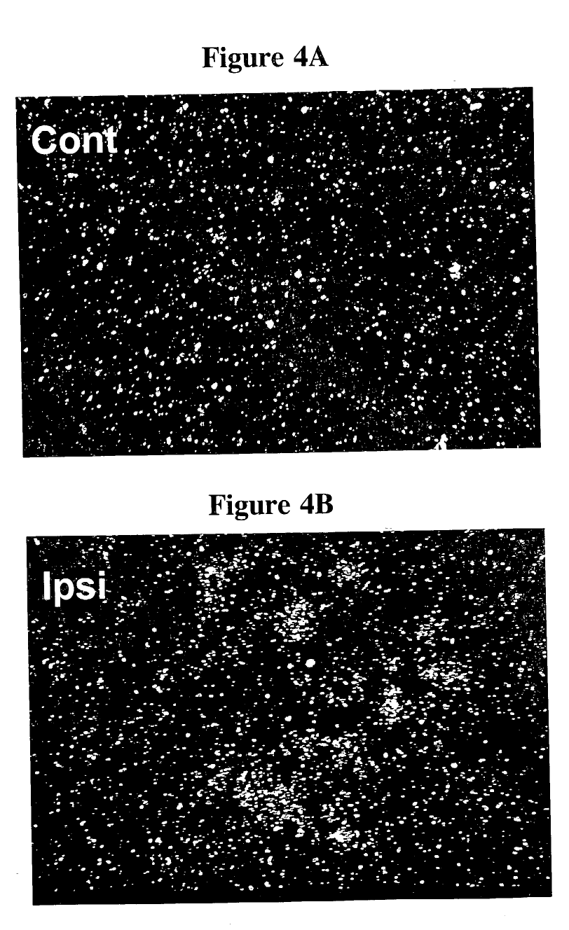

Radio-isotopic ISH analysis of adult rat lumbar spinal cord (FIG. 4) demonstrates the up-regulation of FLRT-3 mRNA in the ipsilateral (Ipsi) large motor neurons 3 days after sciatic cut. Spinal cord from the contralateral (uncut) (Cont) side is shown for comparison.

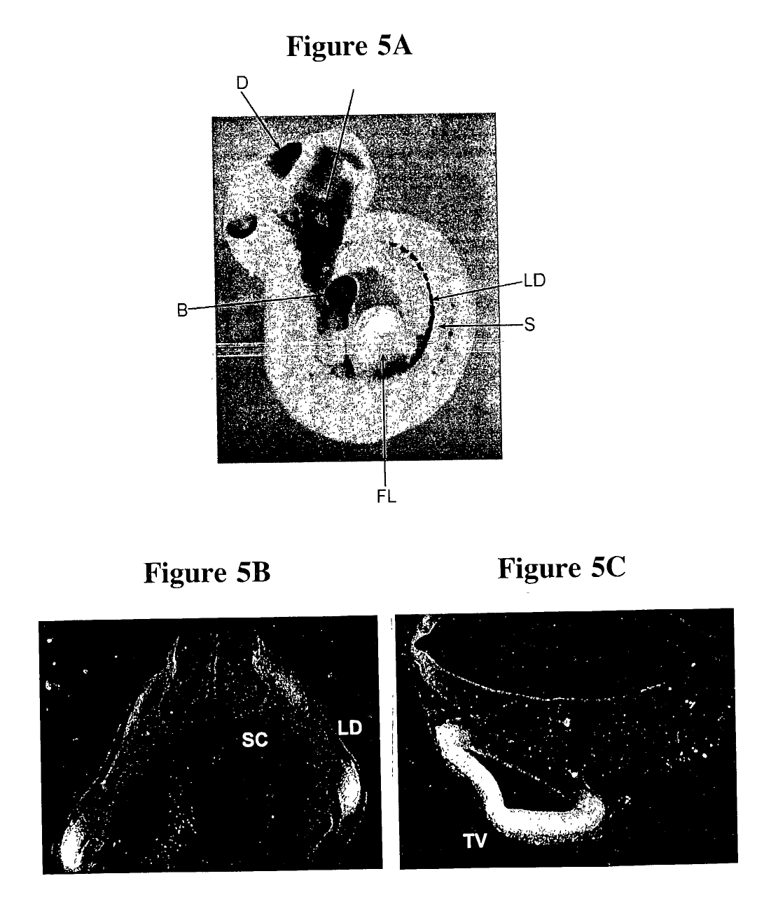

The results of the wholemount ISH using a DIG-labelled riboprobe are shown in FIG. 5A which demonstrates FLRT-3 mRNA expression in E12 rat (B, branchial arch; D, diencephalon; E, eye; FL, forelimb; LD, lip of the dermamyotome; S, somites). FIGS. 5B and 5C show, in dark field, sections through the embryo in A [B) FLRT-3 mRNA expression in the lateral lip of the dermamyotome, LD (SC, spinal cord) and (C) in the telencephalic vesicle, TV].

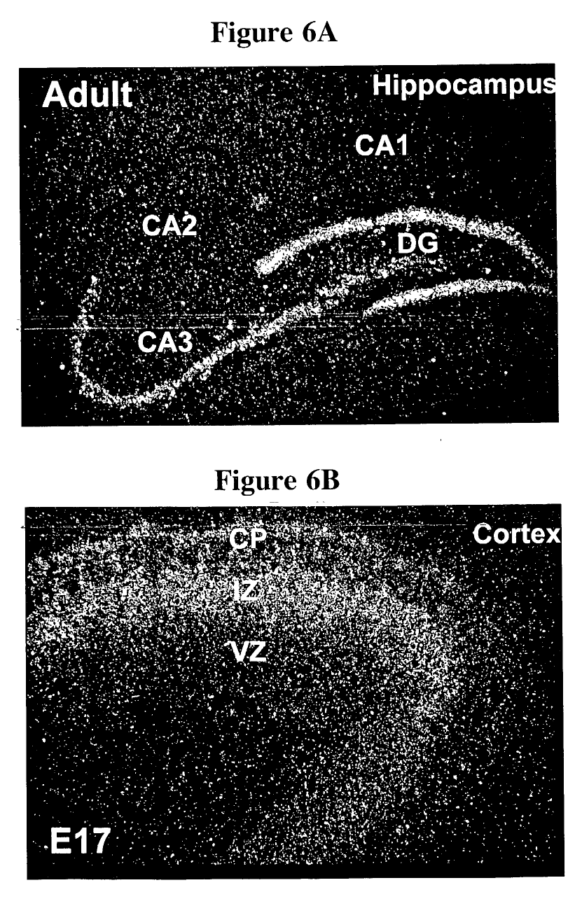

Radio-isotopic ISH demonstrating FLRT-3 mRNA expression in rat brain is shown in FIG. 6. FIG. 6A shows adult rat brain; expression is restricted to the hippocampal area (DG, dentate gyrus). FIG. 6B shows E17 rat brain; expression is particularly strong in the cortex (CP, cortical plate; IZ, intermediate zone; VZ, ventricular zone).

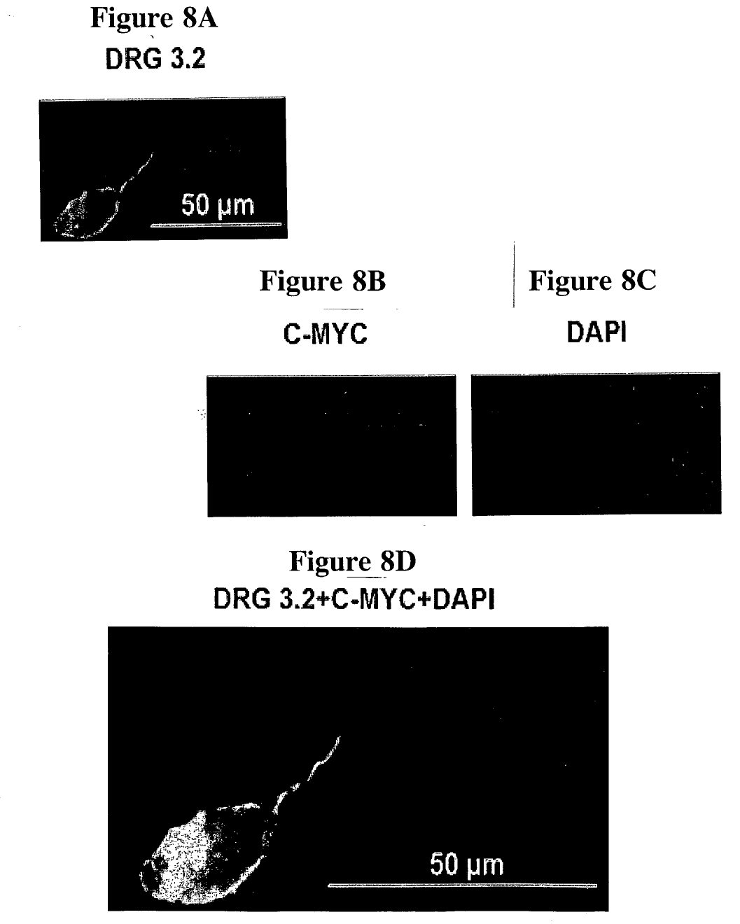

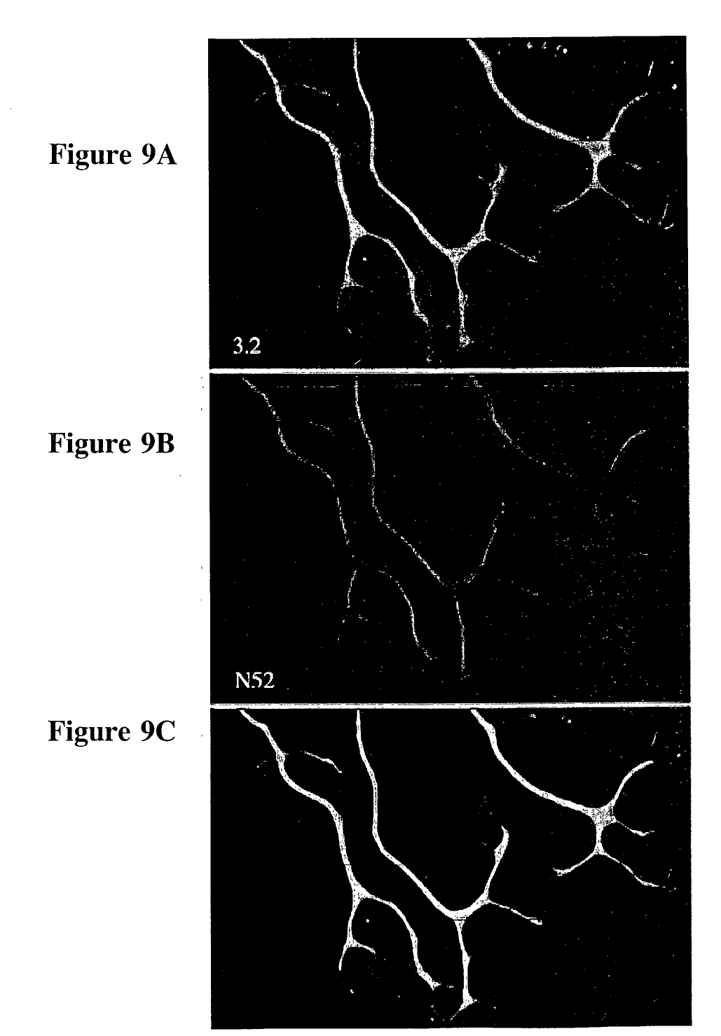

Membrane localisation of FLRT-3 is clearly observed after immunostaining in COS-7 cells transfected with a construct bearing full-length Myc-tagged FLRT-3 (FIG. 8). Double-label immunostaining illustrated in FIG. 9 shows FLRT-3 expression (Ai) and neurofilament N52 expression (ii) in a cultured adult rat DRG neuronal cell. Aiii shows the superimposed image. There is additional expression of FLRT-3 in the growth cones.

In situ hybridisation (ISH) studies and Northern blot analysis verify that after sciatic cut the FLRT-3 gene is strongly up-regulated in most DRG neurons and less strongly in the motor neurons of the spinal cord in both rat and mouse. This up-regulation appears to be peculiar to nerve injury, as northern blot analysis has shown that it does not result from peripheral inflammation caused by injection of complete Freund's adjuvant (CFA) into the rat hind paw. ISH studies have shown that the gene is regulated during embryonic development in rat and mouse, appearing within the developing cortex, midbrain, and segmentally within the somites from E12. Expression persists within the hippocampus and cerebellum of the adult brain.

### Example 3

### Knockdown of FLRT-3 Protein

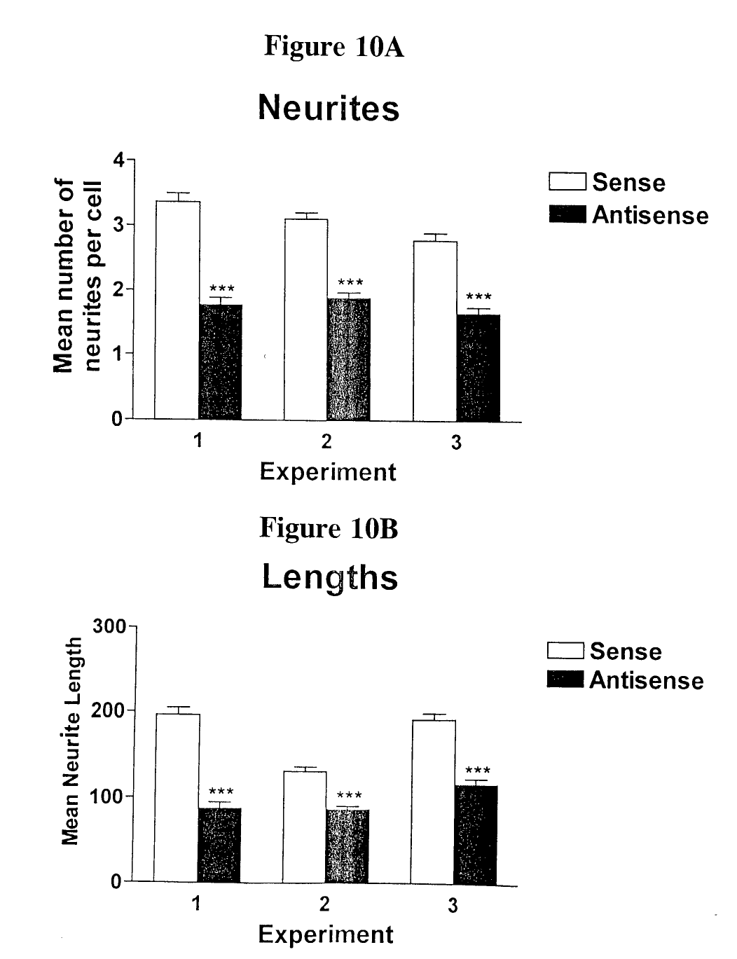

Knockdown of FLRT-3 protein in cultured dissociated adult rat DRG by the application of antisense oligonucleotide produces a decrease in neurite length of approximately 44% and a decrease in neurite number per cell of approximately 43%, relative to control cultures with sense oligonucleotide added (FIG. 10). These decreases are significant (P<0.001, 2-way ANOVA). Prior to statistical analysis, data for neurite length were normalised under a logarithmic function, while those for neurite number were normalised under a square-root function. Data were accumulated from 3 separate experiments.

The up-regulation of FLRT-3 expression in DRG after peripheral nerve injury suggests that it may play a role in the promotion of neurite outgrowth during regeneration. The effects on neurite outgrowth of knocking down and overexpressing FLRT-3 in vitro in dissociated adult rat DRG are strong evidence for this. Knockdown of FLRT-3 in these cultured neuronal cells by the application of antisense oligonucleotides produces a significant reduction in both the number of neurites and the length of the longest neurite per cell, in comparison to control cultures in which sense and scrambled oligonucleotide have been added.

### Example 4

Over-Expression of FLRT-3

Over-expression of FLRT-3 protein in cultured dissociated adult rat DRG using a Herpes Simplex virus delivery system produces an increase of approximately 46% in the length of the longest neurite per cell relative to control cultures over-expressing GFP (FIG. 2). The number of neurites per cell increases by approximately 39%. Data for neurite length were normalised under a logarithmic function prior to statistical analysis using an unpaired, 2-tailed T-test which shows that the increase observed is significant (P<0.0001). The increase in neurite length is also significant (P<0.0259, 2-tailed Mann-Whitney test).

### Example 5

### Identification of a Ligand of FLRT-3

FLRT-3 is purified using routine methods known in the art and is immobilised by attachment to the bottom of the wells of a microtiter plate.

FLRT-3 is radiolabelled using routine methods known in the art and added to the wells of a microtiter plate. Candidate proteins and peptides to be tested are then added to the wells. The reagents are incubated and the wells are washed and counted using a scintillation counter for radioactivity to determine the degree of FLRT-3 binding in the presence of the candidate agents. Binding is tested over a range of concentrations and a series of control “wells” lacking one or more elements of the assays are used for accuracy in evaluating the results.

A ligand of FLRT-3 is identified that binds FLRT-3 and increases the regenerative potential of the neuron.

### Example 6

### Identification of an Agent that Modulates the Activity of FLRT-3

FLRT-3 is purified using routine methods known in the art and is immobilised by attachment to the bottom of the wells of a microtiter plate.

FLRT-3 is radiolabelled using routine methods known in the art and added to the wells of a microtiter plate. Candidate agents to be tested are then be added to the wells. The reagents are incubated and the wells are washed and counted using a scintillation counter for radioactivity to determine the degree of FLRT-3 binding in the presence of the candidate agents. Binding is tested over a range of concentrations and a series of control “wells” lacking one or more elements of the assays are used for accuracy in evaluating the results.

A candidate agent is identified that binds FLRT-3 and increases the activity of FLRT-3.

### Example 7

### Regeneration of Neurons in a Subject in Need Thereof

Using genetic delivery techniques (such as by use of Adenovirus vectors comprising FLRT-3), FLRT-3 is administered to a subject in need of treatment.

### CONCLUSION

FLRT-3 is up-regulated in most adult rat and mouse DRG neurons after sciatic cut and crush but less strongly in the motor neurons of the spinal cord. This up-regulation appears to be injury-specific and is not induced by peripheral inflammation.

FLRT-3 is regulated during embryonic development in rat. It appears within the developing cortex, midbrain, and segmentally within the somites from E12. FLRT-3 expression persists within the hippocampus and cerebellum of adult rat brain.

Protein prediction analysis indicates that FLRT-3 is a single pass transmembrane protein containing LRR's and a Fn III domain, features characteristic of receptors and cell adhesion molecules.

Immunostaining of COS-7 cells transfected with a construct bearing full-length FLRT-3 indicates that FLRT-3 is localised in the membrane.

Antisense knockdown of FLRT-3 in cultured dissociated adult rat DRG indicates that FLRT-3 has a role in the promotion of neurite outgrowth.

Without wishing to be bound theory, we believe that FLRT3 represents a receptor expressed by regenerating neurons that is pro-regenerative and in part responsible for the successful regeneration of sensory neurons. The substantial expression of FLRT3 in the CNS during early development (but its failure to reappear in the adult CNS following damage) indicates that reintroduction of FLRT-3 may be of use in repairing the damaged CNS by driving axonal outgrowth and overcoming inhibitory influences within the external environment. There are also uses for FLRT-3 in neuronal and muscle cell migration.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### REFERENCES

Anderson et al. (1998) Prog Brain Res 117:211-32

Behrens et al. (1999) Nat Genet. 21:326-9.

Bohatschek et al. (2001) Exp Neurol. 172:137-52.

Buchanan & Gay, N.J. (1996) Prog. Bio-phys. Mol. Biol. 65 (1-2): 1-44.

Chaisuksunt et al. (2000a) Neuroscience. 100:87-108.

Chaisuksunt et al. (2000b) J Comp Neurol. 425:382-92.

de Felipe C et al. (1993) Adv Neurol 59:263-71.

Fawcett & Asher (1999) Brain Res Bull 49: 377-391.

Fidler et al. (1999) J Neurosci. 19:8778-88.

Fouad et al. (2001) Brain Res Rev. 36:204-12.

Fournier et al. (2001) Nature. 409:341-6.

Galiano et al. (2001) Eur J Neurosci, 14:327-341

Gold (1999) Drug Metab Rev. 31:649-63

Hull & Bahr M (1994) J Neurobiol. 25:92-105.

Ishii et al. (1994) Pharmacol Ther. 62:125-44.

Jenkins & Hunt (1991) Neurosci Lett. 129:107-10.

Jenkins et al. (1993) Eur J Neurosci. 5:203-9.

Jenkins et al. (1993a) Eur J Neurosci 5:751-9.

Jones et al. (2000) J Comp Neurol 426:468-492

Kalla et al. (2001) J Comp Neurol. 436:182-201.

Kloss et al. (1999) J Comp Neurol 441:162-178

Kobe & Deisenhofer (1994) Trends Biochem. Sci. 19(10): 415-421.

Lacy et al. (1999) Genomics 15, 417-26.

Lilley et al. (2001) J Virol. 75:4343-56.

Livesey et al. (1997) Nature. 390:614-8.

Liu & Strittmatter (2001) Curr Opin Cell Biol. 13:619-26

McKeon et al. (1991) J Neurosci. 11:3398-411

Moon & Fawcett (2001) Eur J Neurosci 14: 1667-1677

Pasterkamp et al. (2001) Eur J Neurosci. 13:457-71.

Raivich et al. (1999) Brain Res Rev 30:77-105

Rong et al (2001) Soc Neurosci Abs 27:901.15

Takahashi et al. (1985) Proc. Natl. Acad. Sci. USA 82(7): 1906-1910.

Qiu et al. (2000) Glia. 29:166-74

Skaper et al. (2001) Prog Neurobiol 65: 593-608.

Vogelezang et al. (2001) J Neurosci 21:6732-6744

Werner et al. (2000) J Neurosci, 20:1822-1830

Werner et al. (2001) Cell Tissue Res 305:25-41

Zhang et al. (2000) Mol Cell Neurosci. 16:71-86.

1. A method of regenerating neurons comprising administering to a subject in need thereof FLRT-3 in amounts effective to cause neuronal regeneration.

2. A method according to claim 1 wherein the neurons are in the central nervous system.

3. A method according to claim 1 wherein FLRT-3 is administered in combination with an agent that modulates FLRT-3.

4. A method according to claim 1 wherein a composition that upregulates FLRT-3 expression is administered to the subject.

5. A method according to claim 4 wherein the composition comprises a vector that delivers a polynucleotide encoding FLRT-3.

6. A method of regenerating neurons comprising administering to a subject in need thereof an agent that modulates FLRT-3 activity in amounts effective to cause neuronal regeneration.

7. A method according to claim 6 wherein the neurons are in the central nervous system.

8. A method according to claim 6 wherein the agent increases the activity of FLRT-3 in central nervous system neurons.

9. A method according to claim 8 wherein the agent upregulates FLRT-3 expression in central nervous system neurons.

10. An assay method for identifying an agent that modulates the activity of FLRT-3 in neurons comprising determining the activity of FLRT-3 in the presence or absence of the agent.

11. An assay method according to claim 10 wherein the neurons are in the central nervous system.

12. An assay method according to claim 10 wherein the assay comprises the step of determining if the agent modulates the ability of FLRT-3 to regenerate neurons.

13. An assay method according to claim 12 wherein the neurons are in the central nervous system.

14. An assay method according to claim 12 wherein the assay comprises the step of determining if the agent increases the activity of FLRT-3 in central nervous system neurons.

15. An agent identified by the assay method according to claim 10.

16. A pharmaceutical composition containing the agent of claim 15.

17. A method of preventing or treating a disease in a subject susceptible to or having the disease, comprising administering to the subject an agent according to claim 10, wherein said agent is capable of modulating the activity of FLRT-3 to cause a beneficial preventative or therapeutic effect.

18. A method according to claim 17 wherein the agent is administered to the central nervous system.

19. A method according to claim 17 wherein the agent increases the activity of FLRT-3.

20. A method according to claim 17 wherein the disease is selected from the group consisting of brain trauma, spinal cord trauma, stroke, cerebral palsy, multiple sclerosis, neuronal migration and muscle cell migration and degenerative diseases such as motor neuron disease, Parkinsons disease and Alzheimers disease.
